(19) United States
(12) Patent Application Publication
(10) Pub. No.: US 2023/0416435 A1
(43) Pub. Date: Dec. 28, 2023

(54) ENHANCING CLUSTERING EFFICIENCY AND KINETICS

(71) Applicants: ILLUMINA, INC., San Diego, CA (US); ILLUMINA CAMBRIDGE LIMITED, Cambridge (GB)

(72) Inventors: Gianluca Andrea Artioli, Cambridge (GB); Timothy J.N. Beech, Cambridge (GB); Mathieu Lessard-Viger, San Diego, CA (US); Rebecca Turk-MacLeod, San Diego, CA (US); Brian D. Mather, San Diego, CA (US); Weixian Xi, San Diego, CA (US); Xavier von Hatten, Cambridge (GB)

(21) Appl. No.: 18/330,298

(22) Filed: Jun. 6, 2023

Related U.S. Application Data
(60) Provisional application No. 63/349,907, filed on Jun. 7, 2022.

(51) Int. Cl. C08F 220/56; C08J 3/075; C12Q 1/6869

(52) U.S. Cl. CPC C08F 220/56; C08J 3/075; C12Q 1/6869; C08J 2333/26

(57) ABSTRACT

A co-polymer includes a plurality of a first monomer including a terminal functional group that is to attach to at least two different primers; a plurality of a second monomer including a second functional group that is different from the terminal functional group, and that is selected from the group consisting of a phenyl group, methoxy propyl, glycosyl, vinyl pyrrolidone, and an imidazole group; and a plurality of a third monomer that is different from the first and second monomers. This co-polymer may be used in a flow cell, and may enhance the clustering efficiency and kinetics.

### CROSS-REFERENCE TO RELATED APPLICATION

This application claims the benefit of U.S. Provisional Application Ser. No. 63/349,907, filed Jun. 7, 2022, the contents of which is incorporated by reference herein in its entirety.

### REFERENCE TO SEQUENCE LISTING

The Revised Sequence Listing submitted herewith is hereby incorporated by reference in its entirety. The name of the file is ILI234B_IP-2282-US_Revised_Sequence_Listing.xml, the size of the file is 15,833 bytes, and the date of creation of the file is Sep. 7, 2023.

### BACKGROUND

Genetic analysis, and in particular nucleic acid sequencing, is taking on increasing importance in modern society, as it has proven useful in a variety of applications. As examples, genetic analysis has been used in predicting a person's risk of contracting some diseases (diagnostics), in determining the probability of therapeutic benefit versus the risk of side effects for a person considering certain treatments (prognostics), and in identifying missing persons, perpetrators of crimes, victims of crimes and casualties of war (forensics). Due to the rapidly increasing demand for reliable genetic information related to an organism, a disease, or an individual, improvements in the throughput of the analysis methods are constantly being investigated.

### SUMMARY

In the examples disclosed herein, nucleic acid sequencing involves the generation of clusters, A cluster is a clonal grouping of a template deoxyribonucleic acid (DNA) strand bound to a flow cell surface. Each cluster is seeded by a single, template DNA strand/construct, which is clonally amplified through bridge amplification until the cluster has multiple copies. Each cluster on the flow cell produces a single sequencing read. A variety of mechanisms to improve the efficiency and/or kinetics of clustering are disclosed herein. The improvement(s) may be due to enhanced availability of the flow cell primers; a reduction in the formation of primer dimers; and/or increased accessibility of the polymeric hydrogel to sequencing proteins and enzymes.

### BRIEF DESCRIPTION OF THE FIGURES

Features of examples of the present disclosure will become apparent by reference to the following detailed description and drawings, in which like reference numerals correspond to similar, though perhaps not identical; components. For the sake of brevity, reference numerals or features having a previously described function may or may not be described in connection with other drawings in which they appear.

FIG. 1A is a top view of an example flow cell;

FIG. 1B is an enlarged, and partially cutaway view of a first example of a flow channel of the flow cell;

FIG. 1C is an enlarged, and partially cutaway view of a second example of a flow channel of the flow cell;

FIG. 1D is an enlarged, and partially cutaway view of a third example of a flow channel of the flow cell;

FIG. 2 is a schematic view of first and second primer sets that can be used in the flow cells disclosed herein;

FIG. 3 is a graph depicting the intensity at 30 minutes (Y axis) versus the primer concentration (μM, X axis) for flow cells with different polymers attached to the surface of the depressions (i.e., nanowells);

FIG. 4 is a graph depicting the number of templates per nanowell (Y axis) versus the number of primers per nanowell (X axis) for a flow cell grafted for 30 minutes and a flow cell grafted for 60 minutes;

FIG. 5 is a graph depicting the number of templates per primer (Y axis) versus the concentration of the primer input (μM, X axis) introduced into respective lanes of flow cells grafted with three different primers (P5/P7, P15/P7, and P15/P7 copper-free);

FIG. 6 is a graft depicting the Cal Fluor Red (CFR) Intensity (reading ×1000, Y axis) versus the grafting concentration (μM, X axis) for flow cells pre-treated with an alkaline buffer and with a carbonate buffer;

FIG. 7 is a graph depicting the mean percentage of clusters passing filter (Y axis) versus the cluster time (minutes, X axis) for flow cells exposed to a pre-treatment and not exposed to a pre-treatment;

FIG. 8 is a graph depicting the mean percentage of depressions that are occupied by a cluster of amplicons (Y axis) versus the cluster time (minutes, X axis) for flow cells exposed to the pre-treatment and not exposed to the pre-treatment;

FIG. 9A through FIG. 9C are graphs depicting the C1 Intensity (Y axis, FIG. 9A), the Q30 score (%, Y axis, FIG. 9B), and the error rate (%, Y axis, FIG. 9C) for first and second reads generated using a flow cell with a standard control lane (i.e., polyethylene glycol) (PEG) in the clustering mix but not attached to the polymer in the flow cell depressions), and a second control lane with no PEG in the clustering mix or attached to the polymer in the flow cell depressions, where three of the flow cell lanes had different concentrations of PEG attached to the polymer in the flow cell depressions;

FIG. 10A through FIG. 10C are graphs depicting the C1 Intensity (Y axis, FIG. 10A), the Q30 score (%, Y axis, FIG. 9B), and the error rate (%, Y axis, FIG. 9C) for first and second reads generated using the flow cell with the standard control lane and the second control lane as described in reference to FIG. 9A through FIG. 9C, where four of the lanes had the same concentration of PEG attached to the polymer in the flow cell depressions but were reacted for different times;

FIG. 11A through FIG. 11D are graphs depicting the single nucleotide polymorphism (SNP) precision (%, Y axis, FIG. 11A), the SNP recall (%, Y axis, FIG. 11B), the insertion-deletion mutation (indel) precision (%, Y axis, FIG. 11C), and the indel recall (%, Y axis, FIG. 11D), for the flow cell with the two control lanes and the three lanes with different concentrations of PEG attached to the polymer; and

FIG. 12A through FIG. 12D are graphs depicting the SNP precision (%, Y axis, FIG. 12A), the SNP recall (%, Y axis, FIG. 12B), the indel precision (%, Y axis, FIG. 12C), and the indel recall (%, Y axis, FIG. 12D), for the flow cell with the two control lanes and the four lanes with the same concentration of PEG attached to the polymer using different reaction times.

### DEFINITIONS

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

An “acrylamide monomer” is a monomer with the structure

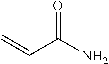

or a monomer including an acrylamide group. Examples of the monomer including an acrylamide group include azido acetamido pentyl acrylamide:

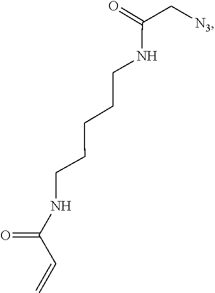 [SMILES: C=CC(=O)NCCCCCNC(=O)CN=[N+]=[N-]]

### N,N-dimethylacrylamide:

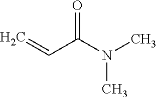

### and N-isopropylacrylamide:

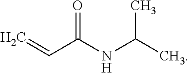

Other acrylamide monomers may be used.

An aldehyde, as used herein, is an organic compound containing a functional group with the structure —CHO, which includes a carbonyl center (i.e., a carbon double-bonded to oxygen) with the carbon atom also bonded to hydrogen and an R group, such as an alkyl or other side chain. The general structure of an aldehyde is:

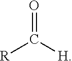

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl,

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amine” or “amino” functional group refers to an —NRaRb group, where Ra and Rb are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocyclyl, as defined herein.

As used herein, the term “attached” refers to the state of two things being joined, fastened, adhered, connected or bound to each other, either directly or indirectly. As examples, bonds that form may be covalent or non-covalent. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, “carbocycle” means a non-aromatic cyclic ring or ring system containing only carbon atoms in the ring system backbone. When the carbocycle is a ring system, two or more rings may be joined together in a fused, bridged or Spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and Spiro[4.4]nonanyl.

As used herein, the term “carboxylic acid” or “carboxyl” as used herein refers to —COOH.

As used herein, “cycloalkylene” means a fully saturated carbocycle ring or ring system that is attached to the rest of the molecule via two points of attachment.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or ‘heterocycloalkyne’ means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a substrate having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

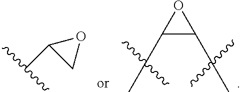

An “epoxy functionalized silsesquioxane” includes a silsesquioxane core that is functionalized with epoxy groups. As used herein, the term “silsesquioxane” refers to a chemical composition that is a hybrid intermediate (RSiO_{1.5}) between that of silica (SiO₂) and silicone (R₂SiO). An example silsesquioxane includes a polyhedral oligomeric silsesquioxane, (commercially available under the tradename POSS® from Hybrid Plastics Inc.). An example of polyhedral oligomeric silsesquioxane can be that described in Kehagias et al., Microelectronic Engineering 86 (2009), pp. 776-778, which is incorporated by reference in its entirety. The composition is an organosilicon compound with the chemical formula [RSiO_{3/2}]ₙ, where n is an even integer ranging from 6 to 14 and at least some of the R groups are epoxy groups. Other R group examples include azide/azido, a thiol, a polyethylene glycol), a norbornene, a tetrazine, acrylates, and/or methacrylates, or further, for example, alkyl, aryl, alkoxy, and/or haloalkyl groups. The resin composition disclosed herein may include one or more different cage or core silsesquioxane structures as monomeric units. In some examples, all of the R groups of the polyhedral oligomeric silsesquioxane are epoxy groups. An example of this type of epoxy functionalized silsesquioxane is glycidyl polyhedral oligomeric silsesquioxane having the structure:

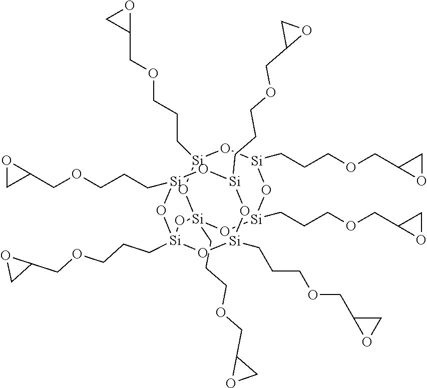

Another example of this type of epoxy functionalized silsesquioxane is epoxycyclohexyl ethyl functionalized polyhedral oligomeric silsesquioxane having the structure:

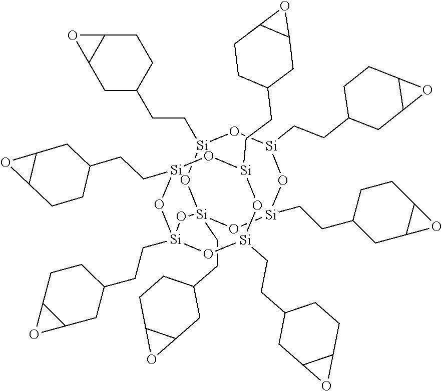

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell enables the detection of the reaction that occurs in the flow channel. For example, the flow cell may include one or more transparent surfaces allowing for the optical detection of arrays, optically labeled molecules, or the like within the flow channel.

As used herein, a “flow channel” or “channel” may be an area defined between two bonded components, which can selectively receive a liquid sample. In some examples, the flow channel may be defined between a substrate and a lid, and thus may be in fluid communication with one or more depressions defined in the substrate. The flow channel may also be defined between two substrate surfaces that are bonded together.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or Spiro-connected fashion. Heterocycles may have any degree of saturation provided that at least one ring in the ring system is not aromatic. In the ring system, the heteroatorn(s) may be present in either a non-aromatic or aromatic ring, The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, “hydroxy” or “hydroxyl” refers to an —OH group.

As used herein, the term “interstitial region” refers to an area, e.g., of a substrate that separates depressions. For example, an interstitial region can separate one depression of an array from another depression of the array. The two depressions that are separated from each other can be discrete, i.e., lacking physical contact with each other. In many examples, the interstitial region is continuous whereas the depressions are discrete, for example, as is the case for a plurality of depressions defined in an otherwise continuous surface. Interstitial regions may have a surface material that differs from the surface material of the depressions defined in the surface. For example, depressions can have a polymeric hydrogel and a primer set therein, and the interstitial regions can be free of the polymeric hydrogel and primer set.

“Nitrile oxide,” as used herein, means a “RₐC≡⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

“Nitrone,” as used herein, means a

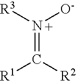

group in which R¹, R², and R³ may be any of the Rₐ and R_{b} groups defined herein, except that R³ is not hydrogen (H).

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

As used herein, the term “primer” is defined as a single stranded nucleic acid sequence (e.g., single strand DNA). Some primers are part of a primer set, which serve as a starting point for template amplification and cluster generation. One example primer set may include “universal” forward and reverse primers, which include sequences capable of annealing to universal primer binding sequences in a template strand/construct. Another example of a primer set may include two different sub-sets of primers. The primer sub-sets are related in that one set includes an un-cleavable first primer and a cleavable second primer, and the other set includes a cleavable first primer and an un-cleavable second primer. These primer sub-sets allow a single template strand to be amplified and clustered across both primer sub-sets, and also enable the generation of forward and reverse strands on the adjacent portions of the polymeric hydrogel due to cleavage groups being present on the opposite primers of the sub-sets. The 5′ terminus of each primer in a primer set may be modified to allow a coupling reaction with a functional group of the polymeric hydrogel. Other primers, referred to herein as sequencing primers, serve as a starting point for DNA synthesis. The length of any of the primers can be any number of bases long and can include a variety of non-natural nucleotides, In an example, the sequencing primer is a short strand, ranging from 10 to 60 bases, or from 20 to 40 bases.

The term “substrate” refers to a structure upon which various components of the flow cell (e.g., polymeric hydrogel, primer(s), etc.) may be added. The substrate may be a wafer, a panel, a rectangular sheet, a die, or any other suitable configuration. The substrate is generally rigid and is insoluble in an aqueous liquid. The substrate may be inert to the chemistry that is present in the depressions. For example, a substrate can be inert to chemistry used to attach the primer(s), used in sequencing reactions, etc. The substrate may be a single layer structure, or a multi-layered structure (e.g., including a support and a patterned resin on the support). Examples of suitable substrates will be described further herein.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refer to five-membered heterocyclic group including four nitrogen atoms. Tetrazole can be optionally substituted.

### Polymeric Hydrogels

The flow cells disclosed herein include a polymeric hydrogel in a lane or in a depression. The polymeric hydrogel is a gel material that can swell when liquid is taken up and can contract when liquid is removed, e.g., by drying. In each of the examples disclosed herein, the polymeric hydrogel is an acrylamide co-polymer. Each example of the acrylamide co-polymer includes at least two different monomers, one of which includes a terminal functional group that is capable of attaching the primers of a primer set to the polymeric hydrogel. Some examples of the acrylamide co-polymer also include another monomer that modulates the hydrogel properties. Other examples of the acrylamide co-polymer include a strained alkyne activated polyethylene glycol (PEG) attached to some of the terminal functional groups. Each of the example polymeric hydrogels will now be described in further detail.

In one example; the acrylamide co-polymeris represented by the following structure (I):

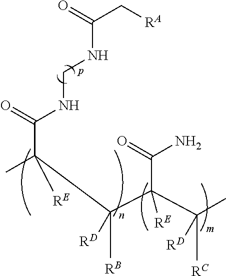

wherein: R^{A} is the terminal functional group that is capable of attaching the primers to the polymeric hydrogel, and is selected from the group consisting of an azide group, an amino group, an alkyne group; an aldehyde group; a hydrazine group, a carboxyl group, a hydroxyl group, a tetrazole group, a tetrazine group, a nitrile oxide group, a nitrone group, a thiol group, and combinations thereof; R^{B}, R^{C}, R^{D}, and R^{E} are each independently selected from the group consisting of H and an alkyl; each of the —(CH₂)ₚ— can be optionally substituted; p is an integer in the range of 1 to 50; n is an integer in the range of 1 to 50,000; and m is an integer in the range of 1 to 100,000. In some instances, the terminal functional group R^{A} may be capable of attaching the primers and may also be capable of attaching the polymeric hydrogel to an underlying substrate. As such, some of the terminal functional group R^{A} may attach the primers and others of the terminal functional group R^{A} may attach to an underlying substrate.

One specific example of the acrylamide co-polymer represented by structure (I) is poly(N-(5-azidoacetamidylpentyl)acrylamide-co-acrylamide, PAZAM.

One of ordinary skill in the art will recognize that the arrangement of the recurring “n” and “m” features in structure (I) are representative, and the monomeric subunits may be present in any order in the polymer structure (e.g., random, block, patterned, or a combination thereof). In some examples, the acrylamide co-polymer in structure (I) is a linear polymer. In some other examples, the acrylamide co-polymer in structure (I) is a lightly cross-linked polymer.

The molecular weight of the acrylamide co-polymer in structure (I) may range from about 5 kDa to about 1500 kDa or from about 10 kDa to about 1000 kDa, or may be, in a specific example, about 312 kDa.

In other examples, the polymeric hydrogel may be a variation of structure (I). In one example, the acrylamide unit may be replaced with N,N-dimethylacrylamide

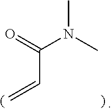

In this example, the acrylamide unit in structure (I) may be replaced with,

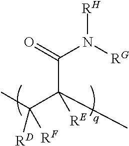

where R^{D}, R^{E}, and R^{F} are each H or a C1-C6 alkyl, and R^{G} and R^{H} are each a C1-C6 alkyl (instead of H as is the case with the acrylamide). In this example, g may be an integer in the range of 1 to 100,000. In another example, the N,N-dimethylacrylamide may be used in addition to the acrylamide unit. In this example, structure (I) may include

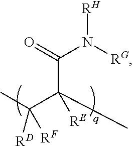

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As still another example of the polymeric hydrogel, the recurring “n” feature in structure (I) may be replaced with a monomer including a heterocyclic azido group having structure (H):

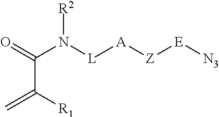

wherein: R₁ is H or a C1-C6 alkyl; R2 is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl,

As still another example, the polymeric hydrogel may include a recurring unit of each of structure (III) and (IV):

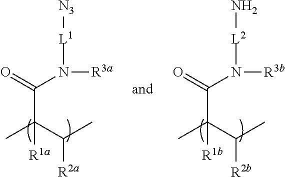

wherein: each of R^{1a}, R^{2a}, R^{1b}, nd R^{2b} is independently selected from H, an alkyl or a phenyl; each of R^{3a} and R^{3b} is independently selected from hydrogen, an alkyl, a phenyl, or a C7-C14 aralkyl; and each L1 and L2 is independently selected from an alkylene linker or a heteroalkylene

It is to be understood that any of the polymeric hydrogels may include a plurality of each of the monomers used to form the hydrogel.

Any of the polymeric hydrogels disclosed herein may further include another monomer in addition to those set forth in structure (I) or in the variations of structure (I), or in addition to structures (III) and (VI). This additional monomer may be selected to modulate the hydrogel properties, such as charge or hydrophilicity, which can contribute to faster clustering kinetics. In these examples, the co-polymer includes a plurality of a first monomer including a terminal functional group that is to attach to at least two different primers; a plurality of a second monomer including a second functional group that is different from the terminal functional group, and that is selected from the group consisting of a phenyl group, methoxy propyl, glycosyl, and an imidazole group; and a plurality of a third monomer that is different from the first and second monomers.

In these examples, the first monomer may be any example of the recurring “n” feature in structure (I) and the third monomer may be any example of the recurring “m” feature in structure (I), In one specific example, the terminal functional group (e.g., R^{A} in structure (I)) is an azide group and the third monomer is an acrylamide.

In particular examples of this co-polymer, the first monomer makes up from about 0.1% to about 20% of the co-polymer; the second monomer makes up from about 0.1% to about 20% of the co-polymer; and the third monomer makes up from about 60% to less than 100% of the co-polymer. In another specific example, the first monomer makes up from about 5% to about 15% of the co-polymer; the second monomer makes up from about 5% to about 15% of the co-polymer; and the third monomer makes up from about 70% to about 90% of the co-polymer.

As noted above, the second monomer includes a functional group that is different from the terminal functional group of the first monomer, and that is selected from the group consisting of a phenyl group, methoxy propyl, glycosyl, vinyl pyrrolidone, and an imidazole group. As specific examples, the second monomer may be an acrylamide monomer selected from the group consisting of N-phenylacrylamide:

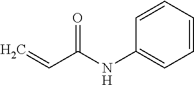

and N-(3-methoxypropyl)acrylamide: [symbol] . As other specific examples, the second monomer may be an acrylate monomer selected from the group consisting of 2-hydroxy-1-methoxypropyl methacrylate:

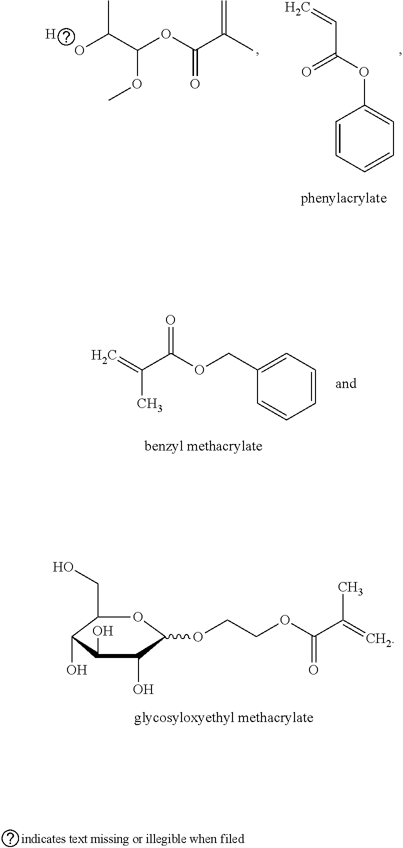

The monomer containing the imidazole may be any vinyl derivative of imidazole, such as

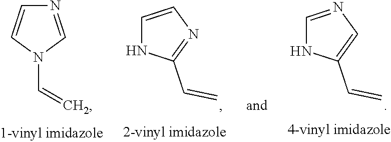

Each of the first, second, and third monomers in this particular example may be incorporated into the co-polymer backbone during polymerization. Suitable polymerization techniques include radical polymerization, reversible addition-fragmentation chain transfer (RAFT) polymerization, atom transfer radical polymerization (ATRP), nitroxide mediated radical (NMP) polymerization in combination with RAFT or ATRP, NMP with an additional cross-linking step, cobalt-mediated polymerization, ionic polymerization, or any other polymerization process that either directly or indirectly yields the desired architecture.

The resulting co-polymer may be a random co-polymer with the first, second, and third monomers randomly positioned along the backbone. In some instances, the monomers may be incorporated statistically along the backbone chain. Polymerization may also be controlled to generate a block copolymer with each block of the backbone including the first, second, or third monomer, or to generate an alternating copolymer with the first, second, and third monomers alternating along the backbone chain.

Alternatively, the second monomer including the phenyl group, methoxy propyl, glycosyl, or the imidazole group may be attached to a side chain of the co-polymer backbone through a coupling reaction (e.g., azide coupling) with the terminal functional group of the polymerized first monomer. This type of attachment will depend upon the other functional group(s) of the second monomer and whether they are capable of undergoing a reaction with the terminal functional group of the first monomer.

The addition of the second monomer (as it is defined in this particular example of the polymeric hydrogel) and/or the third monomer (e.g., dimethylacrylamide) may help to reduce the occurrence of homo-primer dimer formation (e.g., two of the same primers hybridizing to each other) and/or hetero primer dimer formation (e.g., two different primers hybridizing to each other). The reduction in primer dimer formation may be due to a reduction in hydrogen bonding and chain-chain interactions within the polymeric hydrogel. The reduction in primer dimer formation, in turn, leaves more primers available for clustering, which increases the clustering kinetics.

Other examples of the polymeric hydrogel include a strained alkyne activated polyethylene glycol (PEG) attached to some of the terminal functional groups of structure (I) (e.g., R A of the recurring “n” feature in structure (I)) or of the variations of structure (I), or of the co-polymer containing structures (III) and (VI). In these examples, the terminal functional groups may be any functional group that is capable of reacting with (e.g., via a copper-free click reaction) the strained alkyne of the activated polyethylene glycol (PEG). In one specific example, the polymeric hydrogel includes an acrylamide copolymer including terminal azide groups in at least some of the side chains; and a strained alkyne activated polyethylene glycol (PEG) attached to some of the terminal azide groups.

Some specific examples of the strained alkyne activated polyethylene glycol include dibenzocyclooctyne (DBCO) activated polyethylene glycol, monofluorinated cyclooctyne activated polyethylene glycol, difluorinated cyclooctyne activated polyethylene glycol, biarylazacyclooctynone activated polyethylene glycol, and bicyclo[6.1.0]nonyne activated polyethylene glycol. Within each of these examples, a weight average molecular weight of a PEG portion of the strained alkyne activated polyethylene glycol ranges from about 1,000 g/mol to about 20,000 g/mol.

The strained alkyne activated polyethylene glycol may be attached to the terminal functional group of the co-polymer after the co-polymer has been synthesized using any of the polymerization techniques disclosed herein. The strained alkyne activated polyethylene glycol may be added to the co-polymer and the mixture may be exposed to suitable conditions for the click reaction to take place (e.g., in water at about 90° C. for about 2 hours). The strained alkyne activated polyethylene glycol may be incorporated into the co-polymer before it is applied to a flow cell surface, after it is applied to the flow cell surface and before primer grafting takes place, after it is applied to the flow cell surface and simultaneously with primer grafting, or after it is applied to the flow cell surface and after primer grafting takes place.

In an example, a concentration of the strained alkyne activated polyethylene glycol in the polymer hydrogel ranges from about 0.5 mM to about 0.2 mM.

The addition of polyethylene glycol (PEG) to the polymeric hydrogel promotes recombinase binding events and enhances clustering kinetics.

As described herein, some of the terminal functional groups (e.g., R^{A} of structure (I)) of the polymeric hydrogel may attach the primers to the polymeric hydrogel, and others of the terminal functional groups may attach the polymeric hydrogel to an underlying substrate. In other examples, the polymeric hydrogel may include an additional monomer that includes a functional group that is capable of attaching to the underlying substrate. Some examples of substrate-attaching functional groups include an amino group, an alcohol group, an aryl group, and a charged group. Suitable anionic charged groups include sulfates or carboxylic acids. Suitable cationic charged groups include ammonium, guanidinium, or imidazolium. When included, this additional monomer may be incorporated into the co-polymer backbone during polymerization.

### Flow Cells

Any example of the polymeric hydrogel (e.g., structure (I), the co-polymer including three different monomers, the co-polymer including the strained alkyne activated polyethylene glycol (PEG), etc.) described herein may be used in different examples of the flow cell.

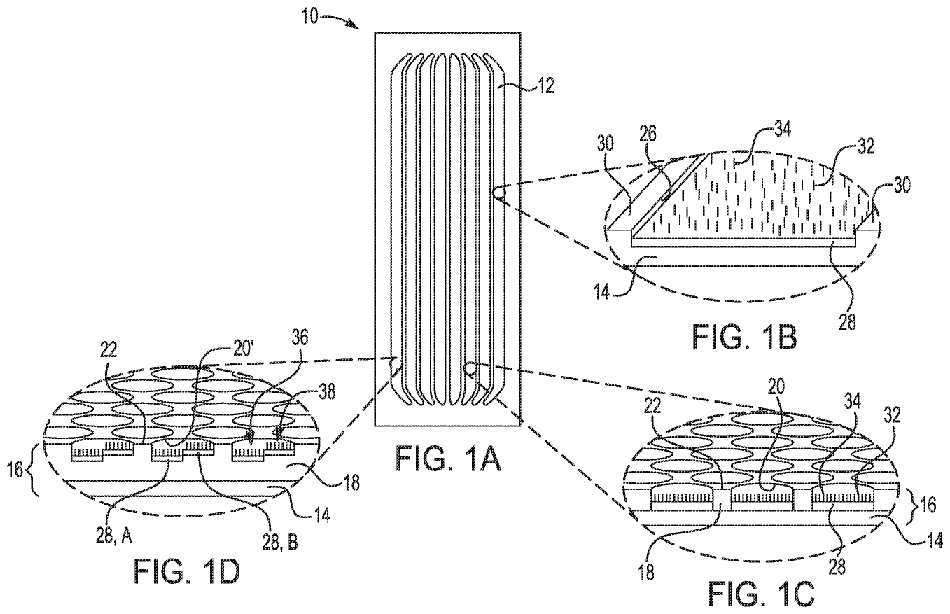

One example of the flow cell 10 is shown in FIG. 1A from a top view. The flow cell 10 may include two patterned or non-patterned structures bonded together, or one patterned or non-patterned structure bonded to a lid.

The patterned structures, the non-patterned structures, or the patterned or non-patterned structure and the lid) may be attached to one another through a spacer layer (not shown). The spacer layer may be any material that will seal portions of the patterned or non-patterned structures together or portions of the patterned or non-patterned structure and the lid. As examples, the spacer layer may be an adhesive, a radiation-absorbing material that aids in bonding, or the like. In some examples, the spacer layer is the radiation-absorbing material, e.g., KAPTON® black. The patterned or non-patterned structures or the patterned structure and the lid may be bonded using any suitable technique, such as laser bonding, diffusion bonding, anodic bonding, eutectic bonding, plasma activation bonding, glass frit bonding, or others methods known in the art.

Defined between the two patterned or non-patterned structures or the one patterned or non-patterned structure and the lid is a flow channel 12. The example shown in FIG. 1A includes eight flow channels 12. While eight flow channels 12 are shown, it is to be understood that any number of flow channels 12 may be included in the flow cell 10 (e.g., a single flow channel 12, four flow channels 12, etc.). Each flow channel 12 may be isolated from each other flow channel 12 so that fluid introduced into a flow channel 12 does not flow into adjacent flow channel(s) 12. Some examples of the fluids introduced into the flow channel 12 may introduce reaction components (e.g., cleaving fluids, DNA sample, polymerases, sequencing primers, nucleotides, etc.), washing solutions, deblocking agents, etc.

The flow channel 12 may have any desirable shape. In an example, the flow channel 12 has a substantially rectangular configuration. The length of the flow channel 12 depends, in part, upon the size of the substrate upon which the patterned or non-patterned structure is formed. The width of the flow channel 12 depends, in part, upon the size of the substrate upon which the patterned or non-patterned structure is formed, the desired number of flow channels 12, the desired space between adjacent channels 12, and the desired space at a perimeter of the patterned or non-patterned structure.

The depth of the flow channel 12 (which does not include the depth of any lane 26 or depression 20, 20′ defined in the patterned or non-patterned structure) can be as small as a monolayer thick when microcontact, aerosol, or inkjet printing is used to deposit a separate material that defines the spacer layer and the walls of the flow channel 12. For other examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than or between the values specified above.

Each flow channel 12 is in fluid communication with an inlet and an outlet (not shown). The inlet and outlet of each flow channel 12 may be positioned at opposed ends of the flow cell 10. The inlets and outlets of the respective flow channels 12 may alternatively be positioned anywhere along the length and width of the flow channel 12 that enables desirable fluid flow.

The inlet allows fluids to be introduced into the flow channel 12, and the outlet allows fluid to be extracted from the flow channel 12. Each of the inlets and outlets is fluidly connected to a fluidic control system (including, e.g., reservoirs, pumps, valves, waste containers, and the like) which controls fluid introduction and expulsion.

FIG. 1B, FIG. 1C, and FIG. 1D depict different examples of the architecture within the flow channel 12.

Each of the architectures includes a substrate, such as a single layer base support 14 (as shown in FIG. 1B), or a multi-layered structure 16 (as shown in FIG. 1C and FIG. 1D).

Examples of suitable single layer base supports 14 include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chernours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

Examples of the multi-layered structure 16 include the base support 14 and at least one other layer 18 thereon, as shown in FIG. 1C and FIG. 1D.

Some examples of the multi-layered structure 16 include glass or silicon as the base support 14, with a coating layer (e.g., layer 18) of tantalum oxide (e.g., tantalum pentoxide or another tantalum oxide(s) (TaOₓ)) or another ceramic oxide at the surface. The coating layer may be patterned using any of the examples disclosed herein.

Other examples of the multi-layered structure 16 include the base support 14 (e.g., glass, silicon, tantalum pentoxide, or any of the other base support 14 materials) and a patterned resin as the other layer 18. It is to be understood that any material that can be selectively deposited, or deposited and patterned to form depressions 20 and interstitial regions 22 (FIG. 1C) or multi-depth depressions 20′ and interstitial regions 22 (FIG. 1D) may be used for the patterned resin.

As one example of the patterned resin, an inorganic oxide may be selectively applied to the base support 14, e.g., via vapor deposition, aerosol printing, or inkjet printing, to define the pattern in the layer 18. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

As another example of the patterned resin, a polymeric resin may be applied to the base support 14 and then patterned to define the pattern in the layer 18. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, etc. Some examples of suitable resins include a polyhedral oligomeric silsesquioxane based resin (e.g., epoxy functionalized silsesquioxanes), a non-polyhedral oligomeric silsesquioxane epoxy resin, a polyethylene glycol) resin, a polyether resin (e.g., ring opened epoxies), an acrylic resin, an acrylate resin, a methacrylate resin, an amorphous fluoropolymer resin (e.g., CYTOP® from BeIlex), and combinations thereof.

In an example, the single base support 14 (whether used singly or as part of the multi-layered structure 16) may be a circular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters), For example, a die may have a width ranging from about 0.1 mm to about 10 mm. While example dimensions have been provided, it is to be understood that a single base support 14 with any suitable dimensions may be used.

The architecture shown in FIG. 1B is a non-patterned structure. The substrate of the non-patterned structure may be the single layer base support 14. In this example, the single layer base support 14 has a lane 26 surrounded by edge regions 30. The lane 26 may be formed using any suitable techniques, such as photolithography, nanoimprint lithography (NIL), stamping techniques, embossing techniques, molding techniques, microetching techniques, etc. The lane 26 provides a designated area for the polymeric hydrogel 28. The edge regions 30 provide bonding regions where two non-patterned structures can be attached to one another or where one non-patterned structure can be attached to a lid. As such, in this example, the surface of the flow cell 10 is non-patterned, and the polymeric hydrogel 28 is positioned within the lane 26 of the non-patterned surface,

To introduce the polymeric hydrogel 28 into the lane 26, a mixture of the polymeric hydrogel 28 may be generated and then applied to the single layer base support 14. In one example, any example of the polymeric hydrogel 28 disclosed herein may be present in a mixture (e.g., with water or with ethanol and water). The mixture may then be applied to the respective substrate surfaces (including in the lane 26) using spin coating, or dipping or dip coating, or flow of the material under positive or negative pressure, or another suitable technique. These types of techniques blanketly deposit the polymeric hydrogel 28 in the lane 26 and on the edge regions 30. Other selective deposition techniques (e.g., involving a mask, controlled printing techniques, etc.) may be used to specifically deposit the polymeric hydrogel 28 in the lane 26 and not on the edge regions 30.

In some examples, the surface of the single layer base support 14 (including the lane 26) may be activated, and then the mixture (including the polymeric hydrogel 28) may be applied thereto. In one example, a silane or silane derivative (e.g., norbornene silane) may be deposited on the surface of the single layer base support 14 using vapor deposition, spin coating, or other deposition methods. In another example, the substrate surface may be exposed to plasma ashing to generate surface-activating agent(s) (e.g., —OH groups) that can adhere to the polymeric hydrogel 28.

Depending upon the chemistry of the polymeric hydrogel 28, the applied mixture may be exposed to a curing process. In an example, curing may take place at a temperature ranging from room temperature (e.g., about 25° C.) to about 95° C. for a time ranging from about 1 millisecond to about several days.

When a blanket deposition technique used, polishing may then be performed in order to remove the polymeric hydrogel 28 from the edge regions 30 at the perimeter of the lane 26, while leaving the polymeric hydrogel 28 on the surface in the lane 26 at least substantially intact.

The architectures shown in FIG. 1C and FIG. 1D depict different examples of a patterned structure. The substrate of each of these patterned structures is the multi-layered structure 16 with depressions 20 or multi-depth depressions 20′ defined in the layer 18. The layer 18 may be selectively deposited, or deposited and patterned to form the depression 20 or the multi-depth depressions 20′. The depressions 20, 20′ provide a designated area for the polymeric hydrogel 28. In the example of FIG. 1C, the surface of the flow cell 10 is patterned with depressions 20 separated by interstitial regions 22, and the polymeric hydrogel 28 is positioned within each depression 20 of the patterned surface. In the example of FIG. 1D, the surface of the flow cell 10 is patterned with multi-depth depressions 20′ separated by interstitial regions 22, and the polymeric hydrogel 28 is positioned within each multi-depth depression 20 of the patterned surface.

Many different layouts of the depressions 20, 20′ may be envisaged, including regular, repeating, and non-regular patterns. In an example, the depressions 20, 20′ are disposed in a hexagonal grid for dose packing and improved density. Other layouts may include, for example, rectangular layouts, triangular layouts, and so forth. In some examples, the layout or pattern can be an x-y format in rows and columns. In some other examples, the layout or pattern can be a repeating arrangement of the depressions 20, 20′ and the interstitial regions 22. In still other examples, the layout or pattern can be a random arrangement of the depressions 20, 20′ and the interstitial regions 22.

The layout or pattern may be characterized with respect to the density (number) of the depressions 20, 20′ in a defined area. For example, the depressions 20, 20′ may be present at a density of approximately 2 million per mm². The density may be tuned to different densities including, for example, a density of about 100 per mm², about 1,000 per mm², about 0.1 million per mm², about 1 million per mm 2 , about 2 million per mm², about 5 million per mm², about 10 million per mm 2 , about 50 million per mm², or more, or less. It is to be further understood that the density can be between one of the lower values and one of the upper values selected from the ranges above, or that other densities (outside of the given ranges) may be used. As examples, a high density array may be characterized as having depressions 20, 20′ separated by less than about 100 nm, a medium density array may be characterized as having the depressions 20 separated by about 400 nm to about 1 μm, and a low density array may be characterized as having the depressions 20, 20′ separated by greater than about 1 μm.

The layout or pattern of the depressions 20, 20′ may also or alternatively be characterized in terms of the average pitch, or the spacing from the center of one depression 20, 20′ to the center of an adjacent depression 20, 20′ (center-to-center spacing) or from the right edge of one depression 20, 20′ to the left edge of an adjacent depression 20, 20′ (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern can be between one of the lower values and one of the upper values selected from the ranges above. In an example, the depressions 20, 20′ have a pitch (center-to-center spacing) of about 1.5 μm. While example average pitch values have been provided, it is to be understood that other average pitch values may be used.

The size of each depression 20, 20′ may be characterized by its volume, opening area, and/or diameter or length and width. For example, the volume can range from about 1×10^{-3 μ}m³ to about 100 μm³, e.g., about 1×10^{-2 μ}m³, about 0.1 μm³, about 1 μm³, about 10 μm³, or more, or less. For another example, the opening area can range from about 1×10^{-3 μ}m² to about 100 μm², e.g., about 1×10^{-2 μ}m², about 0.1 μm², about 1 μm², at least about 10 μm², or more, or less. For yet another example, the diameter or each of the length and width can range from about 0.1 μm to about 100 μm, e.g., about 0.5 μm, about 1 μm, about 10 μm, or more, or less.

The size of each depression 20, 20′ may be also or alternatively be characterized by its depth(s). The depth of the depressions 20 can range from about 0.1 μm to about 100 μm, e.g., about 0.5 μm, about 1 μm, about 10 μm, or more, or less. Each multi-depth depression 20′ includes a deep portion that is adjacent to a shallow portion (as shown in FIG. 1D). It is to be understood that the depth of the deep portion and the depth of the shallow portion are each within the ranges provided for the depression 20, with the caveat that the depth of the deep portion is greater than the depth of the shallow portion. It is to be understood that the height of the internal wall separating the deep and shallow portions will vary depending upon the different depths of the deep and shallow portions. In some examples, it is desirable that the height of the internal wall be substantially equivalent to (e.g., +/−5%) the depth of the shallow portion, thus making the depth of the deep portion about 2 times the depth of the shallow portion.

Any example of the polymeric hydrogel 28 disclosed herein may be used in the architectures shown in FIG. 1C and FIG. 1D.

To introduce the polymeric hydrogel 28 into the depressions 20, 20′, a mixture of the polymeric hydrogel 28 may be generated and then applied to the multi-layered structure 16. In one example, the polymeric hydrogel 28 may be present in a mixture (e.g., with water or with ethanol and water). The mixture may then be applied to the respective substrate surfaces (including in depressions 20, 20′) using spin coating, or dipping or dip coating, or flow of the material under positive or negative pressure, or another suitable technique. These types of techniques blanketly deposit the polymeric hydrogel 28 in the depressions 20, 20′ and on the interstitial regions 22. Other selective deposition techniques (e.g., involving a mask, controlled printing techniques, etc.) may be used to specifically deposit the polymeric hydrogel 28 in the depressions 20, 20′ and not on the interstitial regions 22.

In some examples, the surface of the layer 18 (including the depressions 20′) may be activated, and then the mixture (including the polymeric hydrogel 28) may be applied thereto. In one example, a silane or silane derivative (e.g., norbornene silane) may be deposited on the surface of the layer 18 using vapor deposition, spin coating, or other deposition methods. In another example, the layer 18 may be exposed to plasma ashing to generate surface-activating agent(s) (e.g., -OH groups) that can adhere to the polymeric hydrogel 28.

Depending upon the chemistry of the polymeric hydrogel 28, the applied mixture may be exposed to a curing process. In an example, curing may take place at a temperature ranging from room temperature (e.g., about 25° C.) to about for a time ranging from about 1 millisecond to about several days.

When a blanket deposition process is used to apply the polymeric hydrogel 28, polishing may then be performed in order to remove the polymeric hydrogel 28 from the interstitial regions 22, while leaving the polymeric hydrogel 28 on the surface(s) in the depressions 20, 20′ at least substantially intact.

The architectures shown in FIGS. 1B and 1C include a primer set that includes two different primers 32, 34. The primers 32, 34 make up a primer set that is used in sequential paired end sequencing. In sequential paired end sequencing, the respective forward strands that are generated are sequenced and removed, and then the respective reverse strands are generated, sequenced, and removed.

As examples, the primer set may include P5 and P7 primers, P15 and P7 primers, or any combination of the PA primers, the PB primers, the PC primers, and the PD primers set forth herein. As example combinations, the primer set may include any two PA, PB, PC, and PD primers, or any combination of one PA primer and one PB, PC, or PD primer, or any combination of one PB primer and one PC or PD primer, or any combination of one PC primer and one primer PD.

Examples of P5 and P7 primers are used on the surface of commercial flow cells sold by lumina Inc. for sequencing, for example, on HISEQ™, HISEQX™, MISEQ™, MISEQDX™, MIINISEQ™, NEXTSEQ™, NEXTSEQDX™, NOVASEQ™, ISEQ™, GENOME ANALYZER™, and other instrument platforms.

The P5 primer is:

P5: 5′→3′
(SEQ. ID. NO. 1)
AATGATACGGCGACCACCGAGAUCTACAC
The P7 primer may be any of the following:
P7 #1: 5′→3′
(SEQ. ID. NO. 2)
CAAGCAGAAGACGGCATACGAnAT
P7 #2: 5′→3′
(SEQ. ID. NO. 3)
CAAGCAGAAGACGGCATACnAGAT
P7 #3: 5′→3′
(SEQ. ID. NO. 4)
CAAGCAGAAGACGGCATACnAnAT

where “n” is 8-oxoguanine in each of these sequences.
The P15 primer is:

P15: 5′→3′
(SEQ. ID. NO. 5)
AATGATACGGCGACCACCGAGAnCTACAC

where “n” is allyl-T (a thyrnine nucleotide analog having an allyl functionality).
The other primers (PA-PD) mentioned above include:

PA 5′→3′
(SEQ. ID. NO. 6)
GCTGGCACGTCCGAACGCTTCGTTAATCCGTTGAG
CPA (PA) 5′→3′
(SEQ. ID. NO. 7)
CTCAACGGATTAACGAAGCGTTCGGACGTGCCAGC
PB 5′→3′
(SEQ. ID. NO. 8)
CGTCGTCTGCCATGGCGCTTCGGTGGATATGAACT
cPB (PB') 5′→3′
(SEQ. ID. NO. 9)
AGTTCATATCCACCGAAGCGCCATGGCAGACGACG
PC 5′→3′
(SEQ. ID. NO. 10)
ACGGCCGCTAATATCAACGCGTCGAATCCGCAACT
CPC (PC') 5′→3′
(SEQ. ID. NO. 11)
AGTTGCGGATTCGACGCGTTGATATTAGCGGCCGT
PD 5′→3′
(SEQ. ID. NO. 12)
GCCGCGTTACGTTAGCCGGACTATTCGATGCAGC
cPD (PD') 5′→3′
(SEQ. ID. NO. 13)
GCTGCATCGAATAGTCCGGCTAACGTAACGCGGC.

While not shown in the example sequences for PA-PD, it is to be understood that any of these primers may include a cleavage site, such as uracil, 8-oxoguanine, allyl-T, etc. at any point in the strand, as long as the cleavage sites of the primers 32 and 34 are orthogonal (i.e., the cleaving chemistry of the primer 32 is different than the cleaving chemistry for the primer 34, and thus the two primers 32, 34 are susceptible to different cleaving agents).

Each of the primers 32, 34 disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ terminal end of the primers 32, 34 will vary depending upon the chemistry of the polymeric hydrogel 28. As two examples, the 5′ end functional groups may be a terminal alkyne (e.g., hexynyl) or an internal alkyne, where the alkyne is part of a cyclic compound (e.g., bicyclo[6.1.0]nonyne (BON)). The terminal alkynes can attach to azide groups on the polymeric hydrogel 28. In another example, the primers 32, 34 may include an alkene at the 5′ terminus, which can react with reactive thiol groups on the polymeric hydrogel 28. In still other specific examples, succinimidyl (NHS) ester terminated primers may be reacted with amine groups on the polymeric hydrogel 28, aldehyde terminated primers may be reacted with hydrazine groups on the polymeric hydrogel 28, azide terminated primers may be reacted with an alkyne or DBCO (dibenzocyclooctyne) on the polymeric hydrogel 28, or amino terminated primers may be reacted with activated carboxylate groups on the polymeric hydrogel 28.

The primers 32, 34 may be grafted to the polymeric hydrogel 28 before or after the polymeric hydrogel is introduced into the lane 26 or depression 20. Grafting may be accomplished by flow through deposition (e.g., using a temporarily or permanently bound lid), dunk coating, spray coating, puddle dispensing, or by another suitable method. Each of these example techniques may utilize a primer solution or mixture, which may include the primer(s) 32, 34, water, a buffer, and a catalyst. With any of the grafting methods, the primers 32, 34 attach to the reactive groups of the polymeric hydrogel 28 and do not react with the interstitial regions 22.

The architecture shown in FIG. 1D includes a primer set that includes two different sub-sets 36, 38 of primers. While not shown in FIG. 1C, it is to be understood that these primer sub-sets 36, 38 may also be used in the depressions 20 of the architecture shown in FIG. 1C, where one sub-set 36 is attached to one half of the depression 20 and the other sub-set is attached to the other half of the depression 20.

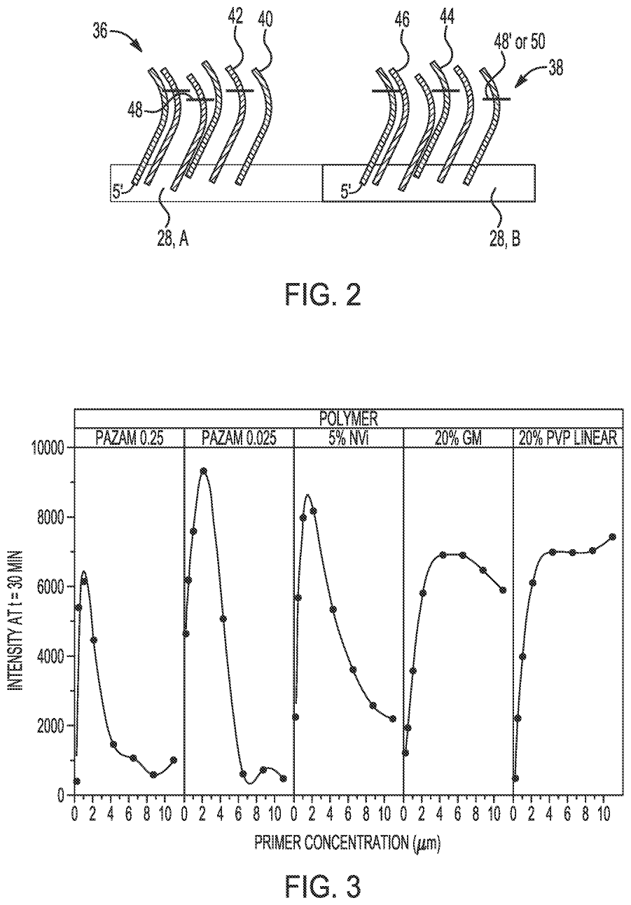

The primer sub-sets 36, 38 (see FIG. 2) enable a simultaneous paired-end read nucleic acid analysis, where forward and reverse strands are sequenced simultaneously. The primer sub-sets 36, 38 enable a cluster of forward strands to be generated in one region A of the polymeric hydrogel 28 and a cluster of reverse strands to be generated in another region B of the polymeric hydrogel 28. As described herein, the primer sub-sets 36, 38 are controlled so that the cleaving (linearization) chemistry is orthogonal at the different polymeric hydrogel regions 28, A and 28, B. More specifically, the primer sub-sets 36, 38 are related in that one sub-set 36 includes an un-cleavable first primer 40 and a cleavable second primer 42, and the other sub-set 38 includes a cleavable first primer 44 and an un-cleavable second primer 46.

The un-cleavable first primer 40 and the cleavable second primer 42 are oligonucleotide pairs, e.g., where the un-cleavable first primer 40 is a forward amplification primer and the cleavable second primer 42 is a reverse amplification primer or where the cleavable second primer 42 is the forward amplification primer and the un-cleavable first primer 40 is the reverse amplification primer, In the first primer sub-set 36, the cleavable second primer 42 includes a cleavage site 48, while the un-cleavable first primer 40 does not include a cleavage site 48.

The cleavable first primer 44 and the un-cleavable second primer 46 are also oligonucleotide pairs, e.g., where the cleavable first primer 44 is a forward amplification primer and the un-cleavable second primer 46 is a reverse amplification primer or where the un-cleavable second primer 46 is the forward amplification primer and the cleavable first primer 44 is the reverse amplification primer. In the second primer sub-set 38, the cleavable first primer 44 includes a cleavage site 48′ or 50, while the un-cleavable second primer 46 does not include a cleavage site 48′ or 50.

It is to be understood that the un-cleavable first primer 40 of the first primer sub-set 36 and the cleavable first primer 44 of the second primer sub-set 38 have the same nucleotide sequence (e.g., both are forward amplification primers), except that the cleavable first primer 44 includes the cleavage site 48′ or 50 integrated into the primer sequence or into a linking molecule attached to the primer sequence. Similarly, the cleavable second primer 42 of the first primer sub-set 36 and the un-cleavable second primer 46 of the second primer sub-set 38 have the same nucleotide sequence (e.g., both are reverse amplification primers), except that the cleavable second primer 42 includes the cleavage site 48 integrated into the primer sequence or into the linking molecule attached to the primer sequence.

It is to be understood that when the first primers 40 and 44 are forward amplification primers, the second primers 42 and 46 are reverse primers, and vice versa.

The un-cleavable primers 40, 46 may be any primer sequence with a universal sequence for capture and/or amplification purposes, such as the P5 and P7 primers, P15 and P7 primers, or any combination of the PA, PD, PC, PD primers (e.g., PA and PB or PA and PD, etc.) without the cleavage site 48, 48′, 50 (e.g., “U” or “n” in some of the sequences set forth herein), In some examples, the P5 and P7 primers are un-cleavable primers 40, 46 because they do not include the cleavage site 48, 48′, 50. It is to be understood that any suitable universal sequence can be used as the un-cleavable primers 40, 46.

Examples of cleavable primers 42, 44 include the P5 and P7 primers, the P15 and P7 primers, or other universal sequence primers (e.g., the PA, PB, PC, PD primers) with the respective cleavage sites 48, 48′, 50 (e.g., “U” or “n” in some of the sequences set forth herein) incorporated into the primer sequence or into the linking molecule attached to the primer sequence. Examples of suitable cleavage sites 48, 48′, 50 include enzymatically cleavable nucleobases or chemically cleavable nucleobases, modified nucleobases, or linkers (e.g., between nucleobases), as described herein, as long they 48 and 48′ or 50 are orthogonal to each other.

As described herein, e.g., in reference to primers 32, 34 and the uracil or cleavage site labeled “n” in the sequences, the cleavage site 48, 48′, 50 of each of the cleavable primers 42, 44 is incorporated into the primer sequence.

In one example, the same type of cleavage site 48, 48′ is used in the cleavable primers 42, 44 of the respective primer sub-sets 36, 38. As an example, the cleavage sites 48, 48′ are uracil bases, and the cleavable primers 42, 44 are P5U and P7U. The uracil bases or other cleavage sites may also be incorporated into any of the PA, PB, PC, and PD primers to generate the cleavable primers 42, 44. In this example, the un-cleavable primer 40 of the oligonucleotide pair 40, 42 may be P7, and the un-cleavable primer 46 of the oligonucleotide pair 44, 46 may be P5. Thus, in this example, the first primer sub-set 36 includes P7, P5U and the second primer sub-set 38 includes P5, P7U. The primers 40, 42 and 44, 46 of the sub-sets 36, 38 have opposite linearization chemistries, which, after amplification, duster generation, and linearization, allows forward template strands to be formed on one polymeric hydrogel region 28, A, and reverse strands to be formed on the other polymeric hydrogel region 28, B.

In another example, different types of cleavage sites 48, 50 are used in the cleavable primers 42, 44 of the respective primer sub-sets 36, 38. As examples, two different enzymatic cleavage sites may be used, two different chemical cleavage sites may be used, or one enzymatic cleavage site and one chemical cleavage site may be used as the linearization cleavage sites 48, 50. Examples of different cleavage sites 48, 50 that may be used in the respective cleavable primers 42, 44 include any combination of the following: vicinal diol, uracil, allyl-T, allyl ether, disulfide, and 8-oxoguanine.

As shown in FIG. 2, each primer sub-set 36 and 38 is attached to a respective region A or B of the polymeric hydrogel 28. The regions 28, A and 28, B are chemically the same, and suitable techniques may be used to sequentially immobilize the primer sub-sets 36, 38 to the desired regions 28, A and 28, B. Alternatively, the primer sub-sets 36, 38 may be pre-grafted to different batches of the polymeric hydrogel 28 and deposited at different times to form the desired regions 28, A and 28, B.

One example of a suitable technique that may be used to attach the primer sub-sets 36, 38 includes the use of a photoresist. In this example, the photoresist is developed to mask one region A while the surface chemistry (e.g., primers 44, 46) is added to the other region B. The photoresist is then removed, and the surface chemistry to the region A under conditions that will not deleteriously affect the region B or its surface chemistry.

Another example of a suitable technique may include the use of a sacrificial layer, such as aluminum. In this example, the sacrificial layer is applied to the substrate (e.g., 14, 16) so that it masks a portion (e.g., one half, the deep portion) of the depression 20, 20′. The other portion (e.g., other half, the shallow portion) of the depression 20, 20′ remains exposed. The exposed portion of the depression 20, 20′ may be activated, e.g., by depositing a suitable silane (e.g., depositing norbornene silane using CVD). In this example, the polymeric hydrogel 28, A is applied over the sacrificial layer and over the exposed and activated portion of the depression 20, 20′. The sacrificial layer is then lifted off, which removes the overlying polymeric hydrogel 28 and exposes the previously covered portion of the depression 20, 20′. The polymeric hydrogel 28, A on the other portion of the depression 20, 20′ remains intact. The primer sub-set 36 may then be grafted to the polymeric hydrogel 24, A. Because the substrate (e.g., 14, 16) has no affinity for the primer sub-set 36, the exposed portion of the depression 20, 20′ is not affected. The substrate (e.g., 14, 16) may then be activated, e.g., by exposure to a solution of norbornene silane. The polymeric hydrogel 28, B may then be applied under conditions (e.g., under high ionic strength (e.g., in the presence of 10×PBS, NaCl, KCl, etc.)) that will not deleteriously affect the region A or its surface chemistry. The primer sub-set 38 may then be grafted to the polymeric hydrogel 28, B.

Still another example of a suitable technique may include pre-grafting the primer sub-sets 36, 38 and sequentially and selectively applying the pre-grafted polymeric materials to the regions A, B.

It is to be understood that while several example methods have been provided, any other methods may be used to immobilize the primer sub-sets 36, 38 to the respective regions A, B.

### Flow Cell Treatments

Any of the flow cells 10 disclosed herein including any of the polymeric hydrogels 28 disclosed herein may be exposed to a method for improving sequencing metrics. These methods may help to loosen the structure of the polymeric hydrogel 28, thus expanding the polymeric hydrogel 28 and enabling better accessibility of the clustering reagents to the primers 32, 34 or the primer sub-sets 36, 38.

One example method for improving sequencing metrics involves pre-treating the flow cell 10 including an example of the primer set grafted to the polymeric hydrogel 28 by introducing water, a basic buffer having a pH ranging from 8 to 11, a high salt buffer, formamide, or isopropyl alcohol to the flow cell 10; increasing the flow cell 10 to a temperature ranging from about 25° C. to about 80° C.; holding the temperature for a time ranging from about 5 minutes to about 2 hours; and then amplifying a library template strand using the grafted primer set (e.g., primers 32, 34 or primer sub-sets 36, 38).

In this example method, the flow cell 10 already has the polymeric hydrogel 28 applied within the lane 26 or depressions 20, 20′ and has the primer set grafted to the polymeric hydrogel 28.

In this example method, the fluid introduced into the flow cell 10 is water, the basic buffer having a pH ranging from 8 to 11, the high salt buffer, formamide, or isopropyl alcohol. Examples of the basic buffer include a borate buffer, N-cyclohexyl-3-aminopropanesulfonic acid (CAPS buffer), 2-Amino-2-methyl-1-propanol buffer (AMP buffer), or N-Cyclohexyl-2-aminoethanesulfonic acid (CHES buffer). Examples of the high salt buffer include water base solutions including a high concentration (above 0.1 M and in the molar range) of a non-acidic and non-basic salt, such as NaCl, MgSO₄, KCl, MgCl₂, in addition to its acid or base pair.

After the fluid is introduced, the temperature of the flow cell 10 is increased, and then the increased temperature is maintained for a time within the given time frame. In one specific example, the temperature of the flow cell 10 is increased to at least 40° C.; and the temperature is held at least 30 minutes. In another specific example, the temperature of the flow cell 10 is increased to about 60° C.; and the temperature is held for about 1 hour.

Holding the flow cell 10 at the increased temperature in the presence of the fluid may help to destroy hydrogen bonds and hydrate/swell the polymeric hydrogel 28. This effectively expands the polymeric hydrogel 28.

Another example method for improving sequencing metrics involves grafting any of the primer sets (e.g., primers 32, 34 or primer sub-sets 36, 38) to the polymeric hydrogel 28 on the flow cell surface in the presence of a carbonate buffer for a time ranging from greater than 30 minutes to about 120 minutes; and amplifying a library template strand using the grafted primer set.

The concentration of the carbonate buffer may be from about 0.1 M to about 1 M. In one example, the carbonate buffer is an aqueous buffer including both sodium carbonate and sodium bicarbonate. The sodium carbonate may range from about 0.01 g/L to about 2.8 g/L and the sodium bicarbonate may range from about 0.09 g/L. to about 2.1 g/L. In one specific example, the carbonate buffer may be prepared by making an aqueous buffer of sodium carbonate (e.g., 1 M); making an aqueous buffer of sodium bicarbonate near saturation (e.g., 1M), and then mixing the sodium carbonate buffer and the sodium bicarbonate buffer together at a volume ratio of 6:1. The final pH may be adjusted to range from about 9 to about 11. In one example, the final pH may be adjusted to range from about 10 to about 10.5 (e.g., 10.3).

In this example, the desired primer set may be introduced into the flow cell 10 (having the polymeric hydrogel 28 already applied within the lane 26 or depressions 20, 20′) with the carbonate buffer. The time for grafting depends upon the temperature of the flow cell 10 and pH of the buffer. The temperature of the flow cell during grafting ranges from about 30° C. to about 80° C. In one example, the temperature ranges from about 40° C. about 60° C. The higher the temperature and the higher the pH, the shorter the grafting time. When the primer sub-sets 36, 38 are used, grafting in the carbonate buffer may take place simultaneously or sequentially as long as the polymeric hydrogel 28 includes regions A, B with different attachment groups for the primer sub-sets 36, 38.

Holding the flow cell 10 at the increased temperature in the presence of the carbonate buffer may help to loosen the structure of the polymeric hydrogel 28. This effectively expands the polymeric hydrogel 28.

### Cluster Generation and Sequencing Methods

Any of the examples of the flow cell 10, which include the polymeric hydrogel 28 and the grafted primer set (e.g., primers 32 and 34 or sub-sets 36, 38) may be used in a sequencing operation.

During sequencing, amplicons of a template strand/construct that is to be sequenced may be formed in the depression 20, 20′ using the primer set immobilized on the polymeric hydrogel 28. At the outset of template strand formation, library fragments/templates may be prepared from any nucleic acid sample (e.g., a DNA sample or an RNA sample). This process occurs off board the flow cell 10. The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers of the primer set. In some examples, the fragments from a single nucleic add sample have the same adapters added thereto. The final library templates include the DNA or cDNA fragment and adapters at both ends. The DNA or cDNA fragment represents the portion of the final library template that is to be sequenced.

A plurality of library templates may be introduced to the flow cell 10. Multiple library templates are hybridized, for example, to one of the primers immobilized on the polymeric hydrogel 28.

Cluster generation may then be performed. In one example of cluster generation, the library templates are copied from the hybridized primers by 3′ extension using a high-fidelity DNA polymerase. The original library templates are denatured, leaving the copies (i.e., amplicons) immobilized on the polymeric hydrogel 28. Isothermal bridge amplification or some other form of amplification may be used to amplify the immobilized copies. For example, the copied templates loop over to hybridize to an adjacent, complementary primer and the polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters. Each cluster of double stranded bridges is denatured. In an example using the primers 32 and 34, the reverse strand is removed by specific base cleavage, leaving forward template strands. Clustering results in the formation of several amplicons immobilized on the polymeric hydrogel 28 (e.g., in the depression 20, 20′ or in areas across the lane 26). This example of clustering is referred to as bridge amplification, and is one example of the amplification that may be performed. It is to be understood that other amplification techniques may be used, such as the exclusion amplification (Examp) workflow (IIlumina Inc.).

Sequencing primers may then be introduced that respectively hybridize to a complementary portion of the sequence of the amplicons. This sequencing primer renders the amplicon ready for sequencing using an incorporation mix.

The incorporation mix may include a plurality of fully functional nucleotides, the polymerase, and a liquid carrier. The liquid carrier of the incorporation mix may be water and/or an ionic salt buffer fluid, such as saline citrate at milli-molar to molar concentrations, sodium chloride, potassium chloride, phosphate buffered saline, etc., and other buffers, such as tris(hydroxymethyl)aminomethane (TRIS) or (4-(2-hydroxyethyl)-1-piperazineethanesulfonic acid) (HEPES). The liquid carrier may also include catalytic metal(s) intended for the incorporation reaction, such as Mg²⁺, Mn²⁺, Ca²⁺, etc. A single catalytic metal or a combination of catalytic metals may be used, and the total amount may range from about 0.01 mM to about 100 mM.

The fully functional nucleotide (FFN) includes the nucleotide, a 3′ OH blocking group attached to the sugar of the nucleotide, and a fluorophore attached to the base of the nucleotide. The nucleotide of the FFN may be any nucleotide describe herein.

The nucleotide of the FFN also includes a 3′ OH blocking group attached thereto, The 3′ OH blocking group may be linked to the 3′ oxygen atom of the sugar molecule in the nucleotide. The 3′ OH blocking group may be a reversible terminator that allows only a single-base incorporation to occur in each sequencing cycle. The reversible terminator stops additional bases from being incorporated into a nascent strand that is complementary to the amplicon. This enables the detection and identification of a single incorporated base. The 3′ OH blocking group can subsequently be removed, enabling additional sequencing cycles to take place at each amplicon. Examples of different 3′ OH blocking groups include a reversible terminator, a 3′-O-allyl reversible terminator (i.e., —CH═CHCH₂), and 3′-O-azidomethyl reversible terminator (i.e., —CH₂ N₃). Other suitable reversible terminators include o-nitrobenzyl ethers, alkyl o-nitrobenzyl carbonate, ester moieties, other allyl-moieties, acetals (e.g., tert-butoxy-ethoxy), MOM (—CHF₂OCH₃) moieties, 2,4-dinitrobenzene sulfenyl, tetrahydrofuranyl ether, 3′ phosphate, ethers, —F, —H₂, —OCH₃, —N₃, —HCOCH₃, and 2-nitrobenzene carbonate.

The nucleotide of the FFN also includes a fluorophore attached to the base of the nucleotide. The fluorophore may be any optically detectable moiety, including luminescent, chemiluminescent, fluorescent, fluorogenic, chromophoric and/or chromogenic moieties. Some examples of suitable optically detectable moieties include fluorescein labels, rhodamine labels, cyanine labels (e.g., Cy3, Cy5, and the like), and the ALEXA® family of fluorescent dyes and other fluorescent and fluorogenic dyes. The fluorophore may be attached to the base of the nucleotide using any suitable linker molecule. In an example, the linker molecule is a spacer group of formula —((CH₂)₂Oₙ— wherein n is an integer between 2 and 50.

In one example, the incorporation mix includes a mixture of different FFNs, which include different bases, e.g., A, T, G, C (as well as U or I). It may also be desirable to utilize a different type of fluorophore for the different FFNs. For example, the fluorophores may be selected so that each fluorophore absorbs excitation radiation and/or emits fluorescence, at a distinguishable wavelength from the other fluorophores. Such distinguishable analogs provide an ability to monitor the presence of different fluorophores simultaneously in the same reaction mixture. In some examples, one of the four FFNs in the incorporation mix may include no fluorophore, while the other three labeled FFNs include different fluorophore.

Any polymerase that can accept the fully functional nucleotide, and that can successfully incorporate the base of the fully functional nucleotide into a nascent strand along the amplicon may be used. Examples polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 9oN (family B), and many others; polymerases from family C, such as Escherichia coli DNA Pol III, and many others, polymerases from family D, such as Pyrococcus furiosus DNA Pol II, and many others; polymerases from family X, such as DNA Pol μ, DNA Pol β, DNA Pol σ, and many others.

In this example method, any example of the incorporation mix is introduced into the flow cell 10, e.g., via the inlet. When the incorporation mix is introduced into the flow cell 10, the mix enters the flow channel 12, and contacts the surface chemistry where the amplicons are present.

The incorporation mix is allowed to incubate in the flow cell 10, and FFNs are incorporated by a polymerase into a nascent strand generated along the amplicon. During incorporation, one of FFNs is incorporated, by a respective polymerase, into one nascent strand that extends one sequencing primer and that is complementary to one of the amplicons. Incorporation is performed in a template strand dependent fashion, and thus detection of the order and type of FFNs added to the nascent strand can be used to determine the sequence of the amplicoll Incorporation occurs in at least some of the amplicons across the depression 20, 20′ or lane 26 during a single sequencing cycle. As such, in at least some of the amplicons across the flow cell 10, respective polymerases extend the hybridized sequencing primer by one of the FFNs in the incorporation mix.

The incorporated FFNs include the reversible termination property due to the presence of the 3′ OH blocking group, which terminates further sequencing primer extension on the nascent strand once the FFN has been added.

After a desired time for incubation and incorporation, the incorporation mix, including at least some non-incorporated FFNs, may be removed from the flow cell 10 during a wash cycle. The wash cycle may involve a flow-through technique, where a washing solution (e.g., a buffer) is directed into, through, and then out of flow channel 12, e.g., by a pump or other suitable mechanism.

Without further incorporation taking place, the most recently incorporated FFNs can be detected through an imaging event. During the imaging event, an illumination system (not shown) may provide an excitation light to the flow cell surfaces containing the surface chemistry. The fluorophore of the incorporated FFNs emit optical signals in response to the excitation light.

After imaging is performed, a cleavage mix may then be introduced into the flow cell 10. In this example, the cleavage mix is capable of i) removing the 3′ OH blocking group from the incorporated FFNs, and ii) cleaving the fluorophore from the FFNs. Examples of 3′ OH blocking groups and suitable de-blocking agents/components in the cleavage mix may include: ester moieties that can be removed by base hydrolysis; allyl-moieties that can be removed with Nal, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II), and 3′ phosphate which can be cleaved by phosphatase enzymes (e.g., polynucleotide kinase). Examples of suitable fluorophore cleaving agents/components in the cleavage mix may include: sodium periodate, which can cleave a vicinal phosphines, such as tris(2-carboxyethyl)phosphine (TCEP) or tri(hydroxypropyl)phosphine (THP), which can cleave azidomethyl linkages; palladium and THP, which can cleave an allyl; bases, which can cleave ester moieties; or any other suitable cleaving agent.

Wash(es) may take place between the various fluid delivery steps. The sequencing cycle can then be repeated n times to extend the sequencing primer by n nucleotides, thereby detecting a sequence of length n. In these examples, paired-end sequencing may be used, where the forward strands are sequenced and removed, and then reverse strands are constructed and sequenced.

Simultaneous paired end sequencing may be used with the primer sub-sets 36, 38. With simultaneous paired end sequencing, during clustering, forward strands are generated on one region A, B of the polymeric hydrogel 28 and reverse strands are generated on the other region B, A of the polymeric hydrogel 28. Incorporation takes place simultaneously at the respective nascent strands of the amplicons being sequenced at both regions A, B, and non-specifically bound FFNs may be present at both regions A, B.

To further illustrate the present disclosure, examples are given herein. It is to be understood that these examples are provided for illustrative purposes and are not to be construed as limiting the scope of the present disclosure.

### EXAMPLES

### Example 1—Terpolymers

Several examples of the co-polymers with three different monomers (i.e., terpolymers) were prepared and compared with one example of the co-polymer of structure (I) (specifically poly(N-(5-azidoacetamidylpentyl)acrylamide-co-acrylamide, PAZAM). The PAZAM concentration in the coating solution was either 0.25 wt % or 0.025 w t%. The monomers in the terpolymers were (azidoacetamidylpentyl)acrylamide, dimethylacrylamide, and one of N-vinylimidazole (5%), glycosyloxyethyl methacrylate (20%), or linear vinyl pyrrolidone (20%).

To test the terpolymers in a flow cell format, a real time i-cBot™ clustering assay (Illumina Inc.) was used. With this assay, the isothermal amplification of DNA in nanowells/depressions (containing one of the polymers and P5/P7 primers crafted thereto) was imaged using EVAGREEN® intercalating dyes (from Biotium) spiked in the EXAMP™ formulation (from Illumina Inc.). From this assay, the maximum intensity (amount of amplified DNA), the slope of the intensity curve (rate of DNA amplification), the time to reach 50% of the maximum intensity (amplification lag time) and the intensity at a specific time (e.g., 30 min) can be determined. The intensity was extracted after 30 minutes of clustering. FIG. 3 illustrates the clustering intensity after 30 minutes (Y axis) versus the primer concentration (X axis, μM). As depicted in FIG. 3, a decrease in clustering kinetics corroborates with an increase in R5/P7 primer density for PAZAM. This may be due to an increase in homo- and/or hetero-dimer formation when a higher number of primers are present. In contrast to PAZAM, each of the terpolymers had higher clustering kinetics at primer concentrations of 6 μM or higher after 30 minutes of clustering. The terpolymers did not appear to be impacted by primer dimer formation.

### Example 2—Carbonate Buffer

A 0.5 M aqueous carbonate buffer was prepared by mixing 1 M solutions of sodium bicarbonate and sodium carbonate at a volume ratio of about 1:6, and then further diluting the buffer with water to achieve the desired polarity. The pH of the carbonate buffer was about 10.

Patterned flow cells with PAZAM in the depressions were used. P5 and P7 primers were introduced into each lane of the flow cell, along with the aqueous carbonate buffer. One flow cell was allowed to incubate for 30 minutes to graft the primers, and another flow cell was allowed to incubate for 60 minutes to graft the primers. In each flow cell, incubation was performed at about 60° C.

After primer grafting, a quantitation assay was performed to compare the grafting processes. To quantify the number of primers per nanowell/depression post-grafting, dye-labelled complementary primers (i.e., fluorescent primers) were hybridized to the surface bound primers and the excess was washed away. The hybridized fluorescent primers were dehybridized, collected, and quantified using a plate reader. The total number of fluorescent primers captured per lane was divided by the number of total numbers of nanowell depressions per lane to obtain the average number of primers per nanowell/depression.

Library fragments (from the PhiX genome) were then introduced and clustering was performed using bridge amplification.

After clustering, a quantitation assay was performed, To quantify the number of templates per nanowell/depression post-clustering, dye-labelled complementary sequences (i.e., fluorescent sequences, which are complementary to the library fragment sequence used to start clustering) were hybridized to the surface bound templates and the excess was washed away. The hybridized fluorescent sequences were dehybridized, collected, and quantified using a plate reader. The total number of fluorescent sequences captured per lane was divided by the number of total numbers of nanowells/depressions per lane to obtain the average number of sequences per nanowell/depression.

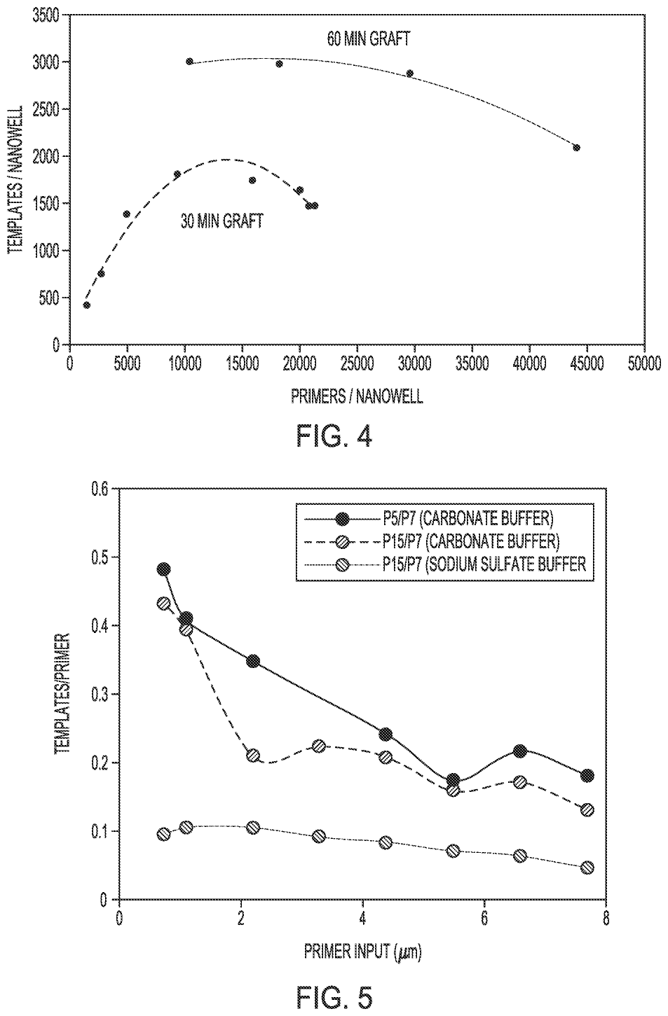

The results from these quantitative assays are shown in FIG. 4. FIG. 4 depicts the number of templates per depression (nanowell) (Y axis) versus the number of primers per depression (nanowell) (X axis) for the 30 minute grafting period and for the 60 minute grafting period. At equivalent primer numbers (e.g., 10,000), the number of templates grafted per depression in the 60 minute period was over 1000 more than the number of templates grafted per depression in the 30 minute period.

The same type of carbonate buffer was then compared with a sodium sulfate buffer (1.5 M), Again, patterned flow cells with PAZAM in the depressions were used. In one flow cell, different concentrations of the P5 and P7 primers were introduced into each lane with the aqueous carbonate buffer. In another flow cell, different concentrations of the P15 and P7 primers were introduced into each lane with the aqueous carbonate buffer. The flow cells containing the carbonate buffer were allowed to incubate at about 60° C. for 60 minutes, and the grafting process was a copper click reaction. In still another flow cell, different concentrations of the P15 and P7 primers were introduced into each lane with the aqueous sodium sulfate buffer. This flow cell was allowed to incubate for 4 hours at about 40° C., and the grafting process was a copper-free click reaction.

After primer grafting, the quantitation assay described herein was performed.

Library fragments (from the PhiX genome) were then introduced and clustering was performed using bridge amplification. After clustering, the quantitation assay described herein was performed.

The results from these quantitative assays are shown in FIG. 5. FIG. 5 depicts the number of templates per primer (Y axis) versus the primer concentration introduced into the respective lanes of the respective flow cells. Cleary, the carbonate buffer resulted in more efficient clustering than the sodium sulfate buffer.

The same type of carbonate buffer was compared with an alkaline buffer solution (commercially available from Sigma-Aldrich, 1.5 M, pH 10.3).

Again, patterned flow cells with PAZAM in the depressions were used. In one flow cell, different concentrations of the P5 and P7 primers were introduced into each lane with the carbonate buffer. In another flow cell, different concentrations of the P5 and P7 primers were introduced into each lane with the alkaline buffer. Both of these flow cells were allowed to incubate for 1 hour at about 60° C.

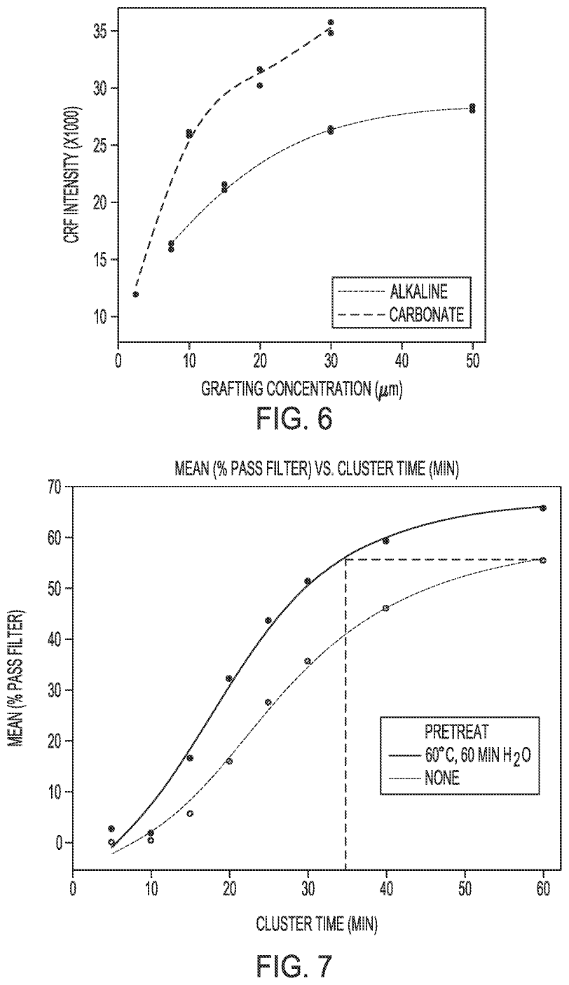

After primer grafting, a Cal Fluor Red (CFR) assay was performed to determine whether primer grafting was successful. During the CFR assay, all lanes of the flow cells were exposed to fluorescently tagged (CAL FLUOR® Red (CFR) dye) oligonucleotides in a buffer solution. These oligonucleotides were complementary to the P5/P7 primers. The fluorescently tagged complementary oligonucleotides bound to surface bound primers and excess CFR tagged complementary oligonucleotides were washed off. The surface was then scanned in a fluorescent detector to measure CFR intensity on the surface. The results are shown in FIG. 6. As depicted, the intensity was higher for the flow cells grafted with the carbonate buffer.

### Example 3—Increased Temperature Soak

Two patterned flow cells were used in this example. Each depression of each flow cell included PAZAM and P5 and P7 primers grafted to the PAZAM.

One of the flow cells was exposed to the following pre-treatment. Before clustering was performed, water was introduced into the flow cell. The temperature of the flow cell was increased to about 60° C. for 1 hour. The other of the flow cells was not exposed to the pre-clustering pretreatment. Library fragments (from the PhiX genome) were then introduced into the pre-treated and non-pre-treated flow cells, and clustering was performed using bridge amplification.

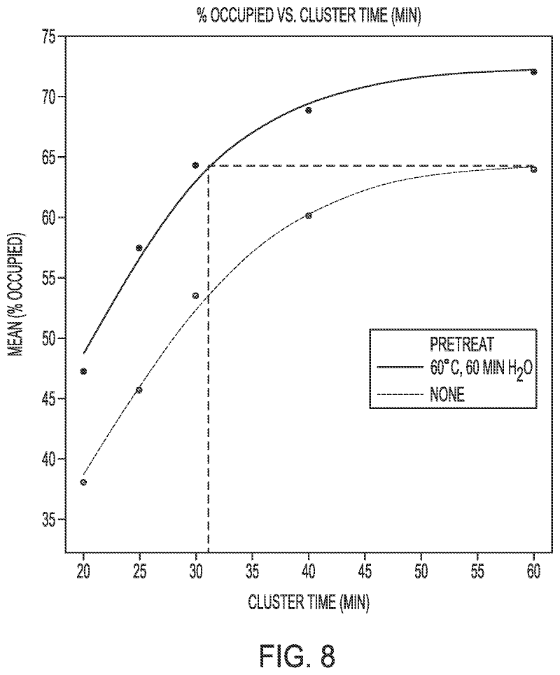

The sequencing data collected included passing filter (% PF) (percentage) and % occupied (or % occupancy). Passing filter (PF) is the metric used to describe dusters which pass a chastity threshold and are used for further processing and analysis of sequencing data. A higher % passing filter result indicates an increased yield of unique dusters used for sequencing data. % Occupied is a quantitative measurement of the percentage of depressions that are occupied by a cluster of amplicons (i.e., the percentage of depressions from which a fluorescence signal is detected, and thus by extension, containing a cluster). The mean % PF results are shown in FIG. 7 as a function of clustering time and the mean % occupied results are shown in FIG. 8 as function of clustering time. As depicted in both FIG. 7 and FIG. 8, the flow cell exposed to the increased temperature pre-treatment process exhibited higher sequencing metrics at similar clustering times.

### Example 4—PEG

A patterned flow cell with PAZAM in the depressions was used. P5 and P7 primers were grafted in each lane of the flow cell for 30 minutes at about 60° C. using a 1.5 μM carbonate buffer (similar to that described in Example 2), In this example, dibenzocyclooctyne (DBCO) activated polyethylene glycol (5,000 g/mol) was introduced into each of the other lanes (2 through 7) to attach the PEG to some of the azide functional groups that remained unattached after primer grafting. Table 1 identifies the lane number, the concentration of DBCO activated PEG in each of the lanes, and the time for the copper-free click reaction to take place.

TABLE 1

| Flow cell lane # | 1* | 2 | 3 | 4 | 5 | 6 | 7 | 8** |
|---|---|---|---|---|---|---|---|---|
| DBCO | N/A | 0.02 | 0.1 | 0.2 | 0.2 | 0.2 | 0.2 | N/A |
| activated PEG | | | | | | | | |
| (mM) | | | | | | | | |
| Reaction Time | N/A | 120 | 120 | 120 | 60 | 30 | 5 | N/A |
| (min) | | | | | | | | |

*std control
**No PEG

As such, both different concentations of DBCO activated PEG and different reaction times were tested in the same flow cell. The copper-free click reaction took place in water at the noted time at about 90° C. In two control lanes (1 and 8) of the flow cell, no PEG was introduced to the PAZAM.

Library fragments (from the Human library (NA12878) and the PhiX genome (1%)) were then introduced into the lanes of the flow cell, The library fragments introduced into lane 1 (“std control”) were introduced in a clustering mix (including surfactants, solvent, polymerase(s), recombinase(s), nucleotide(s), nucleoside triphosphate(s), protein(s), enzyme(s), reducing agent(s), and a source of magnesium) that also included PEG. In contrast, the library fragments introduced into lanes 2-7 with the PEG and into the lane 8 (“No PEG”) were introduced in the clustering mix that did not include PEG. Clustering was performed using bridge amplification.

The HIISEQX™ from lumina Inc, was used to sequence the flow cells. The primary sequencing data collected from the flow cell included C1 intensity, Q30 (%), and error rate (%). The C1 intensity is the fluorescence intensity for the red channel after one sequencing cycle (including read 1 data and read 2 data). Q30 (%) is the percentage of Qscores that were greater than Q30. A Qscore of 30 (Q30) is equivalent to the probability of an incorrect base call 1 in 1000 times. This means that the base call accuracy (i.e., the probability of a correct base call) is 99.9%. A lower base call accuracy of 99% (Q20) will have an incorrect base call probability of 1 in 100, meaning that every 100 base pair sequencing read will likely contain an error. When sequencing quality reaches Q30, virtually all of the reads will be perfect, having 99.9% accuracy. The error rate represents the percentage of incorrect base calls against the PhiX genome.

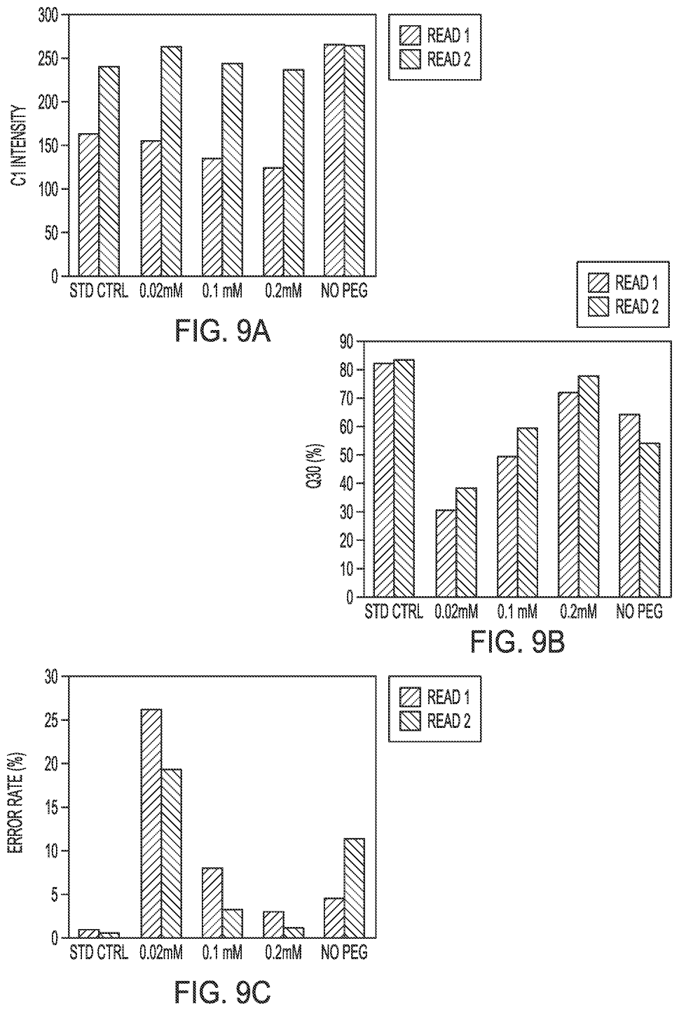

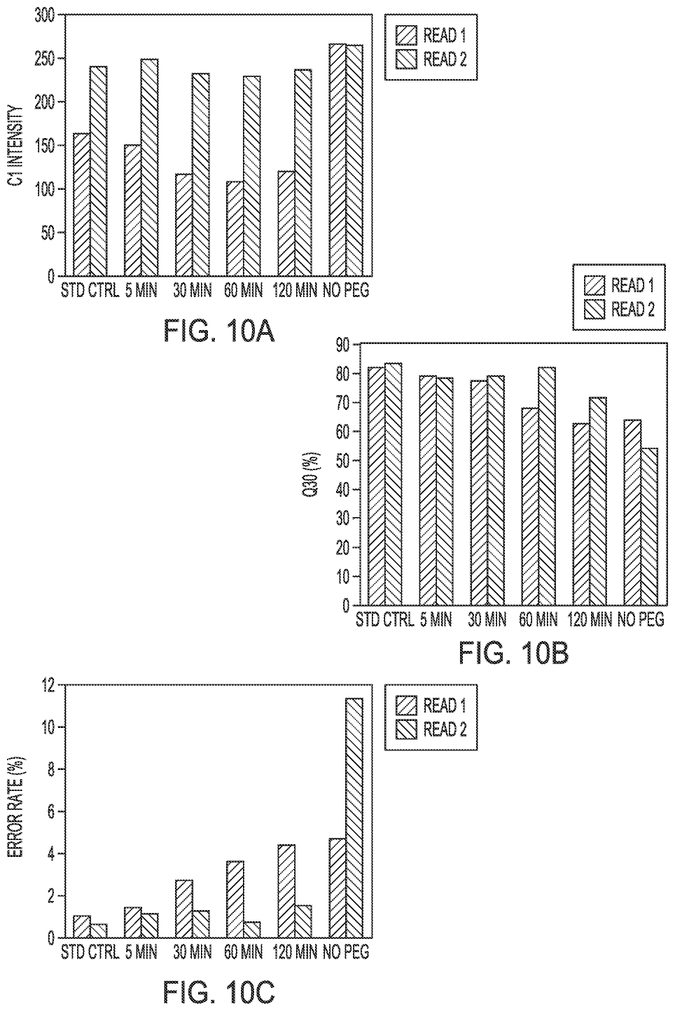

The data in FIG. 9A through FIG. 9C illustrates the respective primary sequencing metrics for lanes 1-4 and 8 of the flow cell, and thus illustrates the data when different concentrations of PEG were introduced. The data in FIG. 10A through FIG. 10C illustrates the respective primary sequencing metrics for lanes 1 and 4-8 the flow cell, and thus illustrates the data when the same concentration of PEG was introduced but reacted for different time periods. The data in FIGS. 9A-9C and 10A-10C illustrate that PEG attached to PAZAM improves the primary sequencing metrics compared to the negative control of lane 8 (i.e., no PEG in the clustering mix and no PEG on the surface). Additionally, the data for the 0.2 mM PEG was comparable to the positive control of lane 1 (i.e., PEG in the clustering mix but not on the surface),

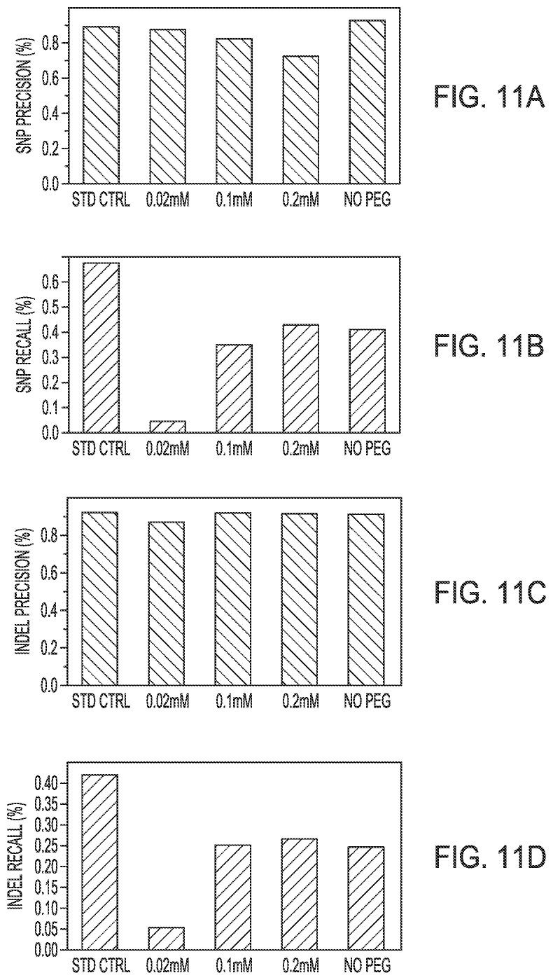

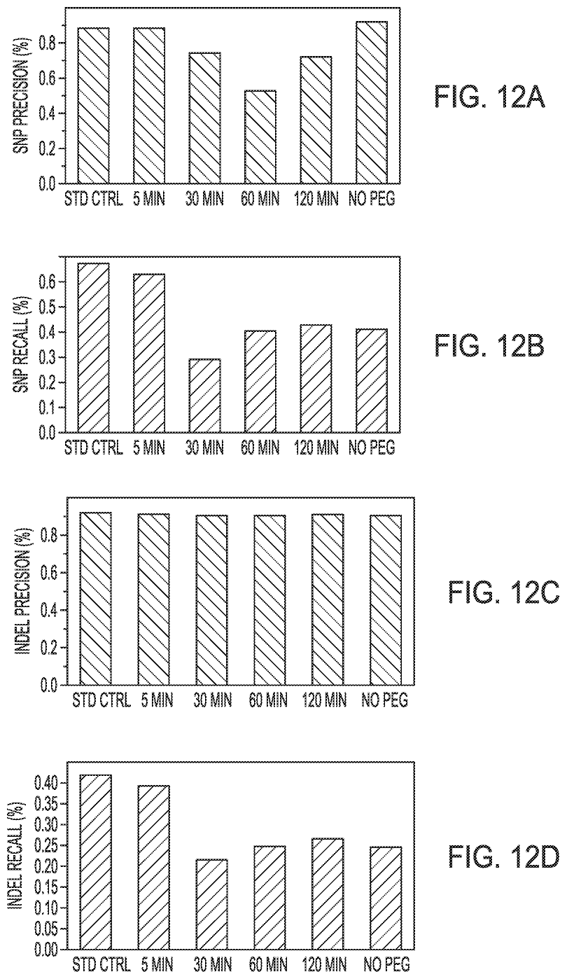

During sequencing, a single nucleotide polymorphism (SNP) changes a single nucleotide in a DNA sequence and an indel incorporates or removes one or more nucleotides. These secondary metrics were also collected, and are shown in FIG. 11A through FIG. 11D and FIG. 12A through FIG. 12D. In these graphs, precision refers to accuracy and is calculated as the ratio of:

[# of True Positive Calls/(# of True Positive Calls+# of False Positive Calls)]

and recall refers to sensitivity and is calculated as the ratio of:

[# of True Positive Calls/(# of True Positive Calls+# of False Negative Calls)]

The results in FIG. 11A through FIG. 12D illustrate that PEG in the clustering mix or attached to the polymer is desirable for recovering secondary metrics.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A co-polymer, comprising:
a plurality of a first monomer including a terminal functional group that is to attach to at least two different primers;
a plurality of a second monomer including a second functional group that is different from the terminal functional group, and that is selected from the group consisting of a phenyl group, methoxy propyl, glycosyl, vinyl pyrrolidone, and an imidazole group; and
a plurality of a third monomer that is different from the first and second monomers.

2. The co-polymer as defined in claim 1, wherein the terminal functional group is selected from the group consisting of an azide group, an amino group, an alkyne group, an aldehyde group, a hydrazine group, a carboxyl group, a hydroxyl group, a tetrazole group, a tetrazine group, a nitrile oxide group, a nitrone group, a thiol group, and combinations thereof.

3. The co-polymer as defined in claim 2, wherein:
the second monomer is an an acrylamide monomer selected from the group consisting of N-phenylacrylamide and N-(3-methoxypropyl)acrylamide; or
the second monomer is an acrylate monomer selected from the group consisting of 2-hydroxy-1-methoxypropyl methacrylate, phenylacrylate, benzyl methacrylate, and glycosyloxyethyl methacrylate;
the second monomer is the monomer containing the imidazole group, and the monomer containing the imidazole group is selected from the group consisting of 1-vinyl imidazole, 2-vinyl imidazole, and 4-vinyl imidazole,

4. The co-polymer as defined in claim 1, wherein:
the terminal functional group is an azide group; and
the third monomer is an acrylamide.

5. The co-polymer as defined in claim 1, wherein:
the first monomer makes up from about 0.1% to about 20% of the co-polymer,
the second monomer makes up from about 0.1% to about 20% of the co-polymer; and
the third monomer makes up from about 60% to less than 100% of the co-polymer.

6. A flow cell, comprising:
a substrate;
a plurality of at least two different primers; and
a co-polymer including:
a plurality of a first monomer including a terminal functional group, wherein at least some of the plurality of at least two different primers are respectively attached to at least some of the terminal functional groups;
a plurality of a second monomer including a second functional group that is different from the terminal functional group, and that is selected from the group consisting of a phenyl group, methoxy propyl, glycosyl, vinyl pyrrolidone, and an imidazole group; and
a plurality of a third monomer that is different from the first and second monomers.

7. The flow cell as defined in claim 6, wherein the terminal functional groups are selected from the group consisting of an azide group, an amino group, an alkyne group, an aldehyde group, a hydrazine group, a carboxyl group, a hydroxyl group, a tetrazole group, a tetrazine group, a nitrile oxide group, a nitrone group, a thiol group, and combinations thereof.

8. The flow cell as defined in claim 7, wherein:
the second monomer is an an acrylamide monomer selected from the group consisting of N-phenylacrylamide and N-(3-methoxypropyl)acrylamide; or
the second monomer is an acrylate monomer selected from the group consisting of 2-hydroxy-1-methoxypropyl methacrylate, phenylacrylate, benzyl methacrylate, and glycosyloxyethyl methacrylate;
the second monomer is the monomer containing the imidazole group, and the monomer containing the imidazole group is selected from the group consisting of 1-vinyl imidazole, 2-vinyl imidazole, and 4-vinyl imidazole.

9. The flow cell as defined in claim 6, wherein:
the substrate includes depressions separated by interstitial regions; and
the co-polymer and the plurality of at least two different primers are positioned within at least some of the depressions.

10. A method for improving sequencing metrics, comprising:
grafting a primer set to a polymeric hydrogel on a flow cell surface in the presence of a carbonate buffer for a time ranging from greater than 30 minutes to about 120 minutes; and
amplifying a library template strand using the grafted primer set.

11. The method as defined in claim 10, wherein the carbonate buffer includes sodium carbonate and sodium bicarbonate.

12. The method as defined in claim 11, wherein a concentration of the sodium carbonate ranges from about 0.01 g/L to about 2.8 g/L and a concentration of the sodium bicarbonate ranges from about 0.09 g/L to about 2.1 g/L.

13. A method for improving sequencing metrics, comprising:
pre-treating a flow cell including a primer set grafted to a polymeric hydrogel by:
introducing water, a basic buffer having a pH ranging from 8 to 11, a high salt buffer, formamide, or isopropyl alcohol to the flow cell;
increasing the flow cell to a temperature ranging from about 25° C. to about 80° C.;
holding the temperature for a time ranging from about 5 minutes to about 2 hours; and
then amplifying a library template strand using the grafted primer set.

14. The method as defined in claim 13, wherein:
the temperature of the flow cell is increased to about 60° C.; and
the temperature is held for about 1 hour.

15. The method as defined in claim 13, wherein:
the temperature of the flow cell is increased to about 40° C.; and
the temperature is held for at least 30 minutes.

16. The method as defined in claim 13, wherein:
the basic buffer or the high salt buffer is introduced; and
the basic buffer is selected from the group consisting of a borate buffer, N-cyclohexyl-3-aminopropanesulfonic acid, 2-Amino-2-methyl-1-propanol buffer, or N-Cyclohexyl-2-aminoethanesulfonic acid; or
the high salt buffer is a water base solution including above 0.1 M of a non-acidic and non-basic salt and its acid or base pair.

17. A polymeric hydrogel, comprising:
an acrylamide co-polymer including terminal azide groups in at least some of the side chains; and
a strained alkyne activated polyethylene glycol (PEG) attached to some of the terminal azide groups.

18. The polymeric hydrogel as defined in claim 17, wherein a concentration of the strained alkyne activated polyethylene glycol in the polymeric hydrogel ranges from about 0.5 mM to about 0.2 mM.

19. The polymeric hydrogel as defined in claim 17, wherein a weight average molecular weight of a PEG portion of the strained alkyne activated polyethylene glycol ranges from about 1,000 g/mol to about 20,000 g/mol.

20. The polymeric hydrogel as defined in claim 17, wherein the strained alkyne activated polyethylene glycol is dibenzocyclooctyne (DBCO) activated polyethylene glycol, monofluorinated cyclooctyne activated polyethylene glycol, difluorinated cyclooctyne activated polyethylene glycol, biarylazacyclooctynone activated polyethylene glycol, and bicyclo[6.1.0]nonyne activated polyethylene glycol.


SEQUENCE LISTING (13 sequences)

SEQ ID NO: 1 - DNA, 29 bp, synthetic construct
  source 1..29: mol_type = other DNA
  modified_base 23: mod_base = OTHER; note = Uracil
aatgatacggcgaccaccgagatctacac

SEQ ID NO: 2 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 3 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 4 - DNA, 24 bp, synthetic construct
  source 1..24: mol_type = other DNA
  modified_base 20: mod_base = OTHER; note = 8-oxoguanine
  modified_base 22: mod_base = OTHER; note = 8-oxoguanine
caagcagaagacggcatacnanat

SEQ ID NO: 5 - DNA, 29 bp, synthetic construct
  source 1..29: mol_type = other DNA
  modified_base 23: mod_base = OTHER; note = allyl-T
aatgatacggcgaccaccgaganctacac

SEQ ID NO: 6 - DNA, 35 bp, synthetic construct
  source 1..35: mol_type = other DNA
gctggcacgtccgaacgcttcgttaatccgttgag

SEQ ID NO: 7 - DNA, 35 bp, synthetic construct
  source 1..35: mol_type = other DNA
ctcaacggattaacgaagcgttcggacgtgccagc

SEQ ID NO: 8 - DNA, 35 bp, synthetic construct
  source 1..35: mol_type = other DNA
cgtcgtctgccatggcgcttcggtggatatgaact

SEQ ID NO: 9 - DNA, 35 bp, synthetic construct
  source 1..35: mol_type = other DNA
agttcatatccaccgaagcgccatggcagacgacg

SEQ ID NO: 10 - DNA, 35 bp, synthetic construct
  source 1..35: mol_type = other DNA
acggccgctaatatcaacgcgtcgaatccgcaact

SEQ ID NO: 11 - DNA, 35 bp, synthetic construct
  source 1..35: mol_type = other DNA
agttgcggattcgacgcgttgatattagcggccgt

SEQ ID NO: 12 - DNA, 34 bp, synthetic construct
  source 1..34: mol_type = other DNA
gccgcgttacgttagccggactattcgatgcagc

SEQ ID NO: 13 - DNA, 34 bp, synthetic construct
  source 1..34: mol_type = other DNA
gctgcatcgaatagtccggctaacgtaacgcggc